SEQUENCE LISTING (104 sequences)

SEQ ID NO: 1 - RNA, 23 nt, Artificial Sequence
  Tnks #1_S_siRNA
cacagagucacacugacuadtdt

SEQ ID NO: 2 - RNA, 23 nt, Artificial Sequence
  Tnks #1_AS_siRNA
uagucagugugacucugugdtdt

SEQ ID NO: 3 - RNA, 23 nt, Artificial Sequence
  Tnks #2_S_siRNA
gucugucguugaguaccuudtdt

SEQ ID NO: 4 - RNA, 23 nt, Artificial Sequence
  Tnks #2_AS_siRNA
aagguacacaacgacagacdtdt

SEQ ID NO: 5 - RNA, 23 nt, Artificial Sequence
  Tnks #3_S_siRNA
acauagcagcguuacugaudtdt

SEQ ID NO: 6 - RNA, 23 nt, Artificial Sequence
  Tnks #3_AS_siRNA
aucaguaacgcugcuaugudtdt

SEQ ID NO: 7 - RNA, 23 nt, Artificial Sequence
  Tnks2 #1_S_siRNA
caguguaguuuugagucuadtdt

SEQ ID NO: 8 - RNA, 23 nt, Artificial Sequence
  Tnks2 #1_AS_siRNA
uagacucaaaacuacacugdtdt

SEQ ID NO: 9 - RNA, 23 nt, Artificial Sequence
  Tnks2 #2_S_siRNA
cuguucugacuggugacuadtdt

SEQ ID NO: 10 - RNA, 23 nt, Artificial Sequence
  Tnks2 #2_AS_siRNA
uagucaccagucagaacagdtdt

SEQ ID NO: 11 - RNA, 23 nt, Artificial Sequence
  Tnks2 #3_S_siRNA
gugucuacuuguaucacaudtdt

SEQ ID NO: 12 - RNA, 23 nt, Artificial Sequence
  Tnks2 #3_AS_siRNA
augugauacaaguagacacdtdt

SEQ ID NO: 13 - RNA, 23 nt, Artificial Sequence
  Ctnnb1 #1_S_siRNA
guuuuaggccuguuuguaadtdt

SEQ ID NO: 14 - RNA, 23 nt, Artificial Sequence
  Ctnnb1 #1_AS_siRNA
uuacaaacaggccuaaaacdtdt

SEQ ID NO: 15 - RNA, 23 nt, Artificial Sequence
  Ctnnb1 #2_S_siRNA
ucugaacgugcauugugaudtdt

SEQ ID NO: 16 - RNA, 23 nt, Artificial Sequence
  Ctnnb1 #2_AS_siRNA
aucacaaugcacguucagadtdt

SEQ ID NO: 17 - RNA, 23 nt, Artificial Sequence
  Ctnnb1 #3_S_siRNA
guaaucuggagacguguaadtdt

SEQ ID NO: 18 - RNA, 23 nt, Artificial Sequence
  Ctnnb1 #3_AS_siRNA
uuacacgucuccagauuacdtdt

SEQ ID NO: 19 - RNA, 23 nt, Artificial Sequence
  Rnf146 #1_S_siRNA
cagauaccuccguugaagadtdt

SEQ ID NO: 20 - RNA, 23 nt, Artificial Sequence
  Rnf146 #1_AS_siRNA
ucuucaacggagguaucugdtdt

SEQ ID NO: 21 - RNA, 23 nt, Artificial Sequence
  Rnf146 #2_S_siRNA
cucuagagcaucacagcuudtdt

SEQ ID NO: 22 - RNA, 23 nt, Artificial Sequence
  Rnf146 #2_AS_siRNA
aagcugugaugcucuagagdtdt

SEQ ID NO: 23 - RNA, 23 nt, Artificial Sequence
  Rnf146 #3_S_siRNA
gucgacaagagauuccugadtdt

SEQ ID NO: 24 - RNA, 23 nt, Artificial Sequence
  Rnf146 #3_AS_siRNA
ucaggaaucucuugucgacdtdt

SEQ ID NO: 25 - RNA, 21 nt, Artificial Sequence
  TNKS_S_siRNA
gcauggagcuuguguuaauuu

SEQ ID NO: 26 - RNA, 21 nt, Artificial Sequence
  TNKS_AS_siRNA
auuaacacaagcuccaugcuu

SEQ ID NO: 27 - RNA, 21 nt, Artificial Sequence
  TNKS2_S_siRNA
ggaaagacguaguugaauauu

SEQ ID NO: 28 - RNA, 21 nt, Artificial Sequence
  TNKS2_AS_siRNA
uauucaacuacgucuuuccuu

SEQ ID NO: 29 - RNA, 23 nt, Artificial Sequence
  Sox9 #1_S_siRNA
guaaaggaagguaacgauudtdt

SEQ ID NO: 30 - RNA, 23 nt, Artificial Sequence
  Sox9 #1_AS_siRNA
aaucguuaccuuccuuuacdtdt

SEQ ID NO: 31 - RNA, 23 nt, Artificial Sequence
  Sox9 #2_S_siRNA
gagacaucggacagaccuudtdt

SEQ ID NO: 32 - RNA, 23 nt, Artificial Sequence
  Sox9 #2_AS_siRNA
aaggucuguccgaugucucdtdt

SEQ ID NO: 33 - RNA, 23 nt, Artificial Sequence
  Sox9 #3_S_siRNA
guuuguuucccucuccaaadtdt

SEQ ID NO: 34 - RNA, 23 nt, Artificial Sequence
  Sox9 #3_AS_siRNA
uuuggagagggaaacaaacdtdt

SEQ ID NO: 35 - DNA, 20 bp, Artificial Sequence
  Hprt_S_Primer
agtcccagcgtcgtgattag

SEQ ID NO: 36 - DNA, 22 bp, Artificial Sequence
  Hprt_AS_Primer
gtatccaacacttcgagaggtc

SEQ ID NO: 37 - DNA, 20 bp, Artificial Sequence
  Tnks1_S_Primer
gaaggaaggagaagttgcgg

SEQ ID NO: 38 - DNA, 22 bp, Artificial Sequence
  Tnks1_AS_Primer
aatgaaaggagaaccgtggaac

SEQ ID NO: 39 - DNA, 20 bp, Artificial Sequence
  Tnks2_S_Primer
cggcgtcttcaacagataca

SEQ ID NO: 40 - DNA, 21 bp, Artificial Sequence
  Tnks2_AS_Primer
agccatcaaccataccttcag

SEQ ID NO: 41 - DNA, 20 bp, Artificial Sequence
  Col2a1_S_Primer
accttggacgccatgaaagt

SEQ ID NO: 42 - DNA, 20 bp, Artificial Sequence
  Col2a1_AS_Primer
cgggaggtcttctgtgatcg

SEQ ID NO: 43 - DNA, 20 bp, Artificial Sequence
  Comp_S_Primer
gtaaacaccgccactgatga

SEQ ID NO: 44 - DNA, 20 bp, Artificial Sequence
  Comp_AS_Primer
tgggagaagcagaagacacc

SEQ ID NO: 45 - DNA, 19 bp, Artificial Sequence
  Col9a2_S_Primer
gatgggtcctcgtggctat

SEQ ID NO: 46 - DNA, 20 bp, Artificial Sequence
  Col9a2_AS_Primer
gttccctttgggcctgttat

SEQ ID NO: 47 - DNA, 22 bp, Artificial Sequence
  Col6a3_S_Primer
ttatggtgctgatgttgactgg

SEQ ID NO: 48 - DNA, 21 bp, Artificial Sequence
  Col6a3_AS_Primer
attgctgttggtttggtcgtt

SEQ ID NO: 49 - DNA, 21 bp, Artificial Sequence
  Acan_S_Primer
cccaagcacagaggtaaacag

SEQ ID NO: 50 - DNA, 20 bp, Artificial Sequence
  Acan_AS_Primer
ctcacattgctcctggtctg

SEQ ID NO: 51 - DNA, 21 bp, Artificial Sequence
  Dcn_S_Primer
aggcttcctactcggctgtga

SEQ ID NO: 52 - DNA, 20 bp, Artificial Sequence
  Dcn_AS_Primer
gttcggcggcatttgacttt

SEQ ID NO: 53 - DNA, 21 bp, Artificial Sequence
  Col6a1_S_Primer
tgaaaatgtgctcctgctgtg

SEQ ID NO: 54 - DNA, 20 bp, Artificial Sequence
  Col6a1_AS_Primer
tgtcccgttgagtgtcagaa

SEQ ID NO: 55 - DNA, 21 bp, Artificial Sequence
  Col9a1_S_Primer
agctgatggattaacaggacc

SEQ ID NO: 56 - DNA, 20 bp, Artificial Sequence
  Col9a1_AS_Primer
ttcccagggtctccaatagg

SEQ ID NO: 57 - DNA, 19 bp, Artificial Sequence
  Bgn_S_Primer
gcattgagatgggcgggaa

SEQ ID NO: 58 - DNA, 20 bp, Artificial Sequence
  Bgn_AS_Primer
agtagggcacagggttgttg

SEQ ID NO: 59 - DNA, 19 bp, Artificial Sequence
  Chad_S_Primer
acaaccgcctgaaccaact

SEQ ID NO: 60 - DNA, 20 bp, Artificial Sequence
  Chad_AS_Primer
ggggagggattctgtgtctt

SEQ ID NO: 61 - DNA, 18 bp, Artificial Sequence
  Matn3_S_Primer
cagtgtgaggggtttctg

SEQ ID NO: 62 - DNA, 20 bp, Artificial Sequence
  Matn3_AS_Primer
agcaccataagttcatagcc

SEQ ID NO: 63 - DNA, 21 bp, Artificial Sequence
  Ctnnb1_S_Primer
ccacaggattacaagaagcgg

SEQ ID NO: 64 - DNA, 20 bp, Artificial Sequence
  Ctnnb1_AS_Primer
ccattcccaccctaccaagt

SEQ ID NO: 65 - DNA, 21 bp, Artificial Sequence
  Rnf146_S_Primer
agcacagagaatgaaccagca

SEQ ID NO: 66 - DNA, 22 bp, Artificial Sequence
  Rnf146_AS_Primer
tgaagcaccctttacacacaga

SEQ ID NO: 67 - DNA, 20 bp, Artificial Sequence
  Sox9_S_Primer
aagatgaccgacgagcagga

SEQ ID NO: 68 - DNA, 20 bp, Artificial Sequence
  Sox9_AS_Primer
atgtgagtctgttccgtggc

SEQ ID NO: 69 - DNA, 19 bp, Artificial Sequence
  HPRT1_S_Primer
cctggcgtcgtgattagtg

SEQ ID NO: 70 - DNA, 21 bp, Artificial Sequence
  HPRT1_AS_Primer
cttgcgaccttgaccatcttt

SEQ ID NO: 71 - DNA, 21 bp, Artificial Sequence
  TNKS1_S_Primer
tcagggaacgattttgctgga

SEQ ID NO: 72 - DNA, 21 bp, Artificial Sequence
  TNKS1_AS_Primer
actctgggtatgcctgttctc

SEQ ID NO: 73 - DNA, 21 bp, Artificial Sequence
  TNKS2_S_Primer
gcgatacccaaggcagacatt

SEQ ID NO: 74 - DNA, 22 bp, Artificial Sequence
  TNKS2_AS_Primer
aacaagagggcagagcagatgg

SEQ ID NO: 75 - DNA, 29 bp, Artificial Sequence
  SOX9_S_Primer
ccgaattcatgaatctcctggaccccttc

SEQ ID NO: 76 - DNA, 29 bp, Artificial Sequence
  SOX9_AS_Primer
cgtctagatcaaggtcgagtgagctgtgt

SEQ ID NO: 77 - DNA, 29 bp, Artificial Sequence
  SOX9_S_Primer
aagaattcgaatctcctggaccccttcat

SEQ ID NO: 78 - DNA, 29 bp, Artificial Sequence
  SOX9_AS_Primer
cgtctagatcaaggtcgagtgagctgtgt

SEQ ID NO: 79 - DNA, 29 bp, Artificial Sequence
  SOX9_S_Primer
aagctagcaaccatggactacaaagacca

SEQ ID NO: 80 - DNA, 29 bp, Artificial Sequence
  SOX9_AS_Primer
cgtctagatcaaggtcgagtgagctgtgt

SEQ ID NO: 81 - DNA, 30 bp, Artificial Sequence
  TNKS2_S_Primer
aaaagcttggatcatgtcgggtcgccgctg

SEQ ID NO: 82 - DNA, 40 bp, Artificial Sequence
  TNKS2_AS_Primer
aaggatccttatccatcgaccataccttcaggcctcataa

SEQ ID NO: 83 - DNA, 23 bp, Artificial Sequence
  SOX9_S_Primer
cagccccctatcgacttccgcga

SEQ ID NO: 84 - DNA, 23 bp, Artificial Sequence
  SOX9_AS_Primer
cccctctcgcttcaggtcagcct

SEQ ID NO: 85 - DNA, 23 bp, Artificial Sequence
  SOX9_S_Primer
agcagcgacgtcatctccaacat

SEQ ID NO: 86 - DNA, 21 bp, Artificial Sequence
  SOX9_AS_Primer
gaagtcgatagggggctgtct

SEQ ID NO: 87 - DNA, 23 bp, Artificial Sequence
  SOX9_S_Primer
agcagcgacgtcatctccaacat

SEQ ID NO: 88 - DNA, 23 bp, Artificial Sequence
  SOX9_AS_Primer
cccctctcgcttcaggtcagcct

SEQ ID NO: 89 - DNA, 19 bp, Artificial Sequence
  SOX9_S_Primer
cccttgccagaggggggca

SEQ ID NO: 90 - DNA, 22 bp, Artificial Sequence
  SOX9_AS_Primer
tgccccctctcgcttcaggtca

SEQ ID NO: 91 - DNA, 22 bp, Artificial Sequence
  SOX9_S_Primer
gacgtggacatcggcgagctga

SEQ ID NO: 92 - DNA, 24 bp, Artificial Sequence
  SOX9_AS_Primer
tgcgaagtcgatagggggctgtct

SEQ ID NO: 93 - DNA, 58 bp, Artificial Sequence
  Control_S_Primer
ccggaaacaagatgaagagcaccaactcgagttggtgctcttcatcttgttttttttg

SEQ ID NO: 94 - DNA, 58 bp, Artificial Sequence
  Control_AS_Primer
aattcaaaaaaaacaagatgaagagcaccaactcgagttggtgctcttcatcttgttt

SEQ ID NO: 95 - DNA, 58 bp, Artificial Sequence
  Tnks_S_Primer
ccgggctagatgtgttggctgatatctcgagatatcagccaacacatctagctttttg

SEQ ID NO: 96 - DNA, 58 bp, Artificial Sequence
  Tnks_AS_Primer
aattcaaaaagctagatgtgttggctgatatctcgagatatcagccaacacatctagc

SEQ ID NO: 97 - DNA, 58 bp, Artificial Sequence
  Tnks2_S_Primer
ccggcatcgacacaagctgattaaactcgagtttaatcagcttgtgtcgatgtttttg

SEQ ID NO: 98 - DNA, 58 bp, Artificial Sequence
  Tnks2_AS_Primer
aattcaaaaacatcgacacaagctgattaaactcgagtttaatcagcttgtgtcgatg

SEQ ID NO: 99 - DNA, 58 bp, Artificial Sequence
  Rnf146_S_Primer
ccggatttctgcccacgtaacattactcgagtaatgttacgtgggcagaaattttttg

SEQ ID NO: 100 - DNA, 58 bp, Artificial Sequence
  Rnf146_AS_Primer
aattcaaaaaatttctgcccacgtaacattactcgagtaatgttacgtgggcagaaat

SEQ ID NO: 101 - DNA, 58 bp, Artificial Sequence
  TNKS_S_Primer
ccgggcccataatgatgtcatggaactcgagttccatgacatcattatgggctttttg

SEQ ID NO: 102 - DNA, 58 bp, Artificial Sequence
  TNKS_AS_Primer
aattcaaaaagcccataatgatgtcatggaactcgagttccatgacatcattatgggc

SEQ ID NO: 103 - DNA, 58 bp, Artificial Sequence
  TNKS2_S_Primer
ccggaaggaaagacgtagttgaatactcgagtattcaactacgtctttcctttttttg

SEQ ID NO: 104 - DNA, 58 bp, Artificial Sequence
  TNKS2_AS_Primer
aattcaaaaaaaggaaagacgtagttgaatactcgagtattcaactacgtctttcctt

(19) United States
(12) Patent Application Publication
(10) Pub. No.: US 2021/0353681 A1
(43) Pub. Date: Nov. 18, 2021

(54) USE OF TNKS INHIBITORS FOR REGENERATION OF CARTILAGE

(71) Applicant: SEOUL NATIONAL UNIVERSITY R&DB FOUNDATION, Seoul (KR)

(72) Inventors: Jin-Hong KIM, Seoul (KR); Sukyeong KIM, Seoul (KR); Sangbin HAN, Gimpo-si (KR); Yongsik CHO, Seoul (KR)

(73) Assignee: SEOUL NATIONAL UNIVERSITY R&DB FOUNDATION, Seoul (KR)

(21) Appl. No.: 15/930,940

(22) Filed: May 13, 2020

(51) Int. Cl. A61K 35/28; A61K 31/7105; C12N 15/113; A61K 38/00; C12N 5/0775

(52) U.S. Cl. CPC A61K 35/28; A61K 31/7105; C12N 5/0668; A61K 38/005; C12N 15/1137

(57) ABSTRACT

The present disclosure relates to a method of treating arthritis by targeting Tankyrase. The methods according to the present disclosure can be advantageously used for regeneration of cartilage tissue and for treating osteoarthritis by maximizing the matrix synthesis in cartilage by inhibition of Tankyrase and regulation of other proteins related therewith.

### BACKGROUND OF THE INVENTION

### Sequence Listing

The Sequence Listing submitted in text format (.txt) filed on May 13, 2020, named “SequenceListing.txt”, created on May 6, 2020 (16.7 KB), is incorporated herein by reference.

### Field of the Invention

The present disclosure is related to the regeneration of cartilage.

### Description of the Related Art

The conventional research related to osteoarthritis (OA) has been focused on the studies identifying the mechanism of degeneration of arthritis. Accordingly, the main factors leading to the degeneration mechanism are well known. Existing treatment strategies also focus on slowing the progression of the disease by suppressing degeneration factors, and these strategies cannot have the fundamental therapeutic effect on regenerating cartilage.

Cartilage tissue is a tissue that gradually degrades when it begins to be damaged by aging or injury. Degenerative arthritis is a disease that is afflicted by 4.41 million people in Korea as of 2014, and the demand for treatment is rapidly increasing. However, drugs used to treat degenerative arthritis remain at pain relief levels such as hyaluronic acid and anti-inflammatory drugs. The treatments that induce fundamental regeneration of cartilage have not yet been developed, and research is in its infancy.

Korean Patent Application Publication No. 2014-0144508 relates to a composition for treating damaged cartilage by regeneration thereof and discloses a composition comprising granulocyte macrophage-colony stimulating factor; GM-CSF as an effective ingredient for treating damaged cartilage by regenerating cartilage.

Korean Patent Application Publication No. 2005-0012226 relates to regeneration of cartilage by use of TGF-beta and chondrocyte and discloses a method of treating osteoarthritis by treating the cells with members of TGF super family.

However, no such documents disclose in connection with the treatment of cartilage regeneration targeting the factor disclosed herein. In order to fundamentally regenerate cartilage that has undergone degeneration, molecular mechanisms that regulate cartilage regeneration factors are needed to be identified, and the development of a treatment strategy through regulation of the factors is required.

### SUMMARY OF THE INVENTION

The present disclosure is to provide a method treating arthritis or related disease through the regeneration of cartilage tissue by maximizing the ability of the chondrocytes to synthesize matrices by regulating Tankyrase-SOX9 pathway.

In one aspect of the present disclosure, a method of treating arthritis in a subject in need thereof comprising the step of administering to the subject an effective amount of an inhibitor of Tankyrase; or a modified adult stem cell in which the expression of Tankyrase is suppressed or Tankyrase gene is knocked out, wherein the inhibitor of Tankyrase or the modified adult stem cell stabilizes the Sox9 protein or increases the concentration of the Sox9 protein by inhibiting the Tankyrase activity promoting the degradation of Sox9 protein.

In one embodiment, the inhibitor of Tankyrase leads to chondrogenic differentiation of an adult stem cells leading to chondrogenic regeneration.

In other embodiment, the inhibitor of Tankyrase is an agent that binds to a nicotinamide sub-domain or region of ARTD domain which is a catalytic domain of a Tankyrase protein, an agent that binds to an adenosine sub-domain of a Tankyrase protein or an agent that binds to an unidentified domain of a Tankyrase protein.

In other embodiment, the agent that binds to a nicotinamide sub-domain of ARTD domain is XAV939 {3,5,7,8-tetrahydro-2-[4-(trifluoromethyl)phenyl]-4H-thiopyrano[4,3-d]pyrimidine-4-one} or MN-66 {2-[4-(1-methylethyl)phenyl]-4H-1-benzopyran-4-one}; the agent that binds to an adenosine sub-domain of a Tankyrase protein is IWR-1 [4-(1,3,3a,4,7,7a-hexahydro-1,3-dioxo-4,7-methano-2H-isoindole-2-yl)-N-8-quinolynyl-benzamide], JW55 {N-[4-[[[[tetrahydro-4-(4-methoxyphenyl)-2H-pyran-4-yl]methyl]amino]carbonyl]phenyl]-2-purancarboxamide}, WIKI4 2-[3-[[4-(4-methoxyphenyl)-5-(4-pyridynyl)-4H-1,2,4-triazol-3-yl]thio]propyl]-1Hbenz[de]isoquinoline-1,3(2H)-dion, TC-E5001 {3-(4-methoxyphenyl)-5-[[[4-(4-methoxyphenyl)-5-methyl-4H-1,2,4-triazol-3-yl]thio]methyl]-1,2,4-oxadiazol or G007-LK {(E)-4-(5-(2-(4-(2-chlorophenyl)-5-(5-(methylsulfonyl)pyridine-2-yl)-4H-1,2,4-triazol-3-yl)vinyl)-1,3,4-oxadiazole-2-yl)benzonitrile}; and the agent that binds to an unidentified domain of a Tankyrase protein is G244-LM {3,5,7,8-tetrahydro-2-[4-[2-(methylsulfonyl) phenyl]-1-piperazynyl]-4H-thiopyrano[4,3-d]pyrimidine-4-one}, or AZ6102 {rel-2-[4-[6-[(3R,5S)-3,5-dimethyl-1-piperazynyl]-4-methyl-3-pyridynyl]phenyl]-3,7-dihydro-7-methyl-4H-pyrrolo[2,3-d]pyrimidine-4-one}, or isomers or derivative thereof.

In other embodiment, the inhibitor is a siRNA that suppresses the expression of Tankyrase gene into a Tankyrase protein.

In other embodiment, the siRNA is a dsRNA consisting of RNAs of SEQ ID NO: X1 and SEQ ID NO:X2; or a dsRNA consisting of RNAs of SEQ ID NO: X3 and SEQ ID NO:X4.

In other embodiment, the adult stem cell is autologous or allogenic.

In other aspect, there is provided a method of promoting the differentiation of an adult stem cell into a cartilage cell by treating the stem cell with an inhibitor of Tankyrase.

### Advantageous Effects

Here it was found that Tankyrase is an upstream regulator of SOX9, which is known as an important factor in the formation of cartilage matrix, and the inhibition of Tankyrase can lead to the cartilage regeneration in vivo and in vitro useful for OA therapies. Here it was also found that the cartilage regeneration is possible by promoting the cartilage matrix protein synthesis of chondrocytes present in cartilage tissues and the differentiation of MSC into chondrocytes. Thus the mechanism identified herein can be advantageously used for treating various disease such as OA which can benefit from the cartilage regeneration by inhibiting Tankyrase.

### BRIEF DESCRIPTION OF THE DRAWINGS

FIGS. 1A to 11 are the results of the identification of Tankyrase as a regulator of cartilage anabolic axis. (a) Heatmap of Pearson correlation coefficients of transcript levels for cartilage matrix genes from 16 BXD mouse strains. (b) Factor loadings plot of 14 cartilage matrix genes in cartilage of the 16 BXD mouse strains in terms of transcript abundance, with Tnks and Tnks2 added to the plot. Pearson's r and P value displayed next to Tnks or Tnks2 point represent correlation strength between Factor 1 and Tnks or Tnks2. (c) Correlation between Tnks or Tnks2 and Col2a1 or Acan mRNA levels in the 16 BXD mouse strains. (d) Knockdown efficiency of various Tnks and Tnks2 siRNAs in primary cultured mouse chondrocytes (n=3). siTnks #2 and siTnks 2 #3 were used throughout this study. e-g mRNA and protein levels of cartilage-specific matrix genes in mouse chondrocytes treated with (e) control or Tnks and Tnks2 siRNAs (n=4) or (f, g) drugs (n=7). Col6a5, Col6a6, and Col13a1 mRNAs were undetected. (h) Gene set enrichment analysis (GSEA) of cartilage-signature genes in mouse chondrocytes transfected with siTnks and siTnks2 compared to control siRNA. (i) GSEA of cartilage-signature genes in chondrocytes treated with tankyrase inhibitors compared to vehicle. Cartilage-signature genes are listed in Table 8. Genes upregulated in mouse chondrocytes compared to mouse embryonic fibroblasts were selected as cartilage-signature genes. (d-g) Data represent means±s.e.m. *P<0.05, **P<0.01, ***P<0.001; by ANOVA.

FIGS. 2A to 2G are the results showing that pro-anabolic effect (confirmation of cartilage matrix gene expression) of tankyrase inhibition is mediated by a β-catenin-independent pathway. (a) TOPFlash β-catenin reporter assay in chondrocytes with activated β-catenin signal pathway after control shRNA or shTnks and shTnks 2 transfection (n=7). (b) Lysates of chondrocytes with activated β-catenin signal pathway were immunoblotted for β-catenin after treatment with control or Tnks and Tnks2 siRNAs. (c) TOPFlash β-catenin reporter assay in chondrocytes with activated β-catenin signal pathway following Tnks inhibitory drug treatment (10 μM, 48 h; n=5). (d) Lysates of chondrocytes with activated β-catenin signal pathway were immunoblotted for β-catenin after treatment with the indicated Tnks inhibitory drugs for 48 h. β-catenin. (e) mRNA levels of cartilage matrix genes in chondrocytes transfected with control, Tnks and Tnks2, or Ctnnb1 siRNAs (n=4). (f) TOPFlash β-catenin reporter assay in chondrocytes with activated β-catenin signal pathway after 24 h of iCRT 14 treatment as a direct inhibitor of β-catenin (n=4). (g) Levels of cartilage matrix proteins in chondrocytes with activated β-catenin signal pathway treated with iCRT 14 for 24 h as a direct inhibitor of β-catenin. (a-d, f) Wnt-3a recombinant protein was added 24 h before harvest. (a, c, e, f) Data represent means±s.e.m. *P<0.05, **P<0.01, ***P<0.001; by ANOVA. * P<0.05, ** P<0.01, *** P<0.001; ANOVA (b, d, g).

FIGS. 3A to 3O are the results showing that Tankyrase interacts with SOX9 and regulates its protein stability and it directly interact with SOX9 and the changes of the concentration of SOX9 when Tankyrase is suppressed. (a) Flowchart of tankyrase substrate identification in chondrocytes. (b) Venn diagram illustrating the overlap of tankyrase-binding proteins identified by three biological replicates (BR) using LC/MS-MS. (c) Histogram of the maximum TTS of the identified tankyrase-binding proteins. ▾ indicates the bin that includes proteins belonging to the chondrogenesis protein set defined by IPA. (d) Heatmap of TTS and disorder score of TBDs from the predicted tankyrase-binding proteins having a maximum TTS of ≥0.385 and belonging to the IPA chondrogenesis protein set. The cutoff of 0.385 is the TTS of the tankyrase-binding motifs of mouse AXIN1 and AXIN2. (e) Coimmunoprecipitation of endogenous TNKS with SOX9 in chondrocytes. (f) in situ proximity ligation assay (PLA) to detect interaction between endogenous TNKS and endogenous SOX9 in primary cultured mouse chondrocytes. Red signals indicate the interactions of endogenous TNKS-SOX9. DAPI was used for counter-staining for nuclei. Scale bar: 25 μm (top), 10 μm (bottom). (g) Pull-down assays of GFP-tagged TNKS or TNKS2 with HA-tagged SOX9 in HEK293T cells. (h) Schematic representation of the predicted TBDs in human SOX9 protein (top) and sequence alignment of TBD1 and TBD2 of SOX9 among vertebrates (bottom). Colored letters indicate the consensus amino acid sequence of TBDs. (i) Superimposition of TNKS2:3BP2 and TNKS2:MCL1 complexes with TNKS2 bound to SOX9-TBD1/2. (j) Pull-down assays of Myc-tagged TNKS2 with HA-tagged wild-type SOX9 or TBD1 or TBD2 deleted SOX9 mutants in HEK293T cells. (k) Pull-down assay of TNKS2 with wild-type or TBD1/2-deleted SOX9 in HEK293T cells. 1 PARylation of wild-type or TBD1/2-deleted SOX9 in HEK293T cells. (l) PARylation assay of wild type or TBD1/2 deleted SOX9 in HEK293T cells. (m) SOX9 immunoblots in chondrocytes after siTnks and siTnks2 treatment. (n) SOX9 immunoblots in chondrocytes after drug treatment. (o) Cycloheximide (CHX) chase analysis of wild-type or TBD1/2-deleted SOX9 in HEK293 cells.

FIGS. 4A to 4G are the results showing that RNF146 does not regulate SOX9 activity and cartilage matrix anabolism. (a) TOPFlash β-catenin reporter assay in chondrocytes with β-catenin signal pathway activated after control shRNA or shRnf146 transfection=5). (b) Lysate of Chondrocyte with β-catenin signal pathway activated were immunoblotted for β-catenin after treatment with control siRNA or siRnf146. (a, b) Recombinant Wnt-3a was added 24 h before harvest. (c) 4×48-p89 SOX9-dependent Col2a1 luciferase reporter assay in chondrocytes transfected with control shRNA, shRnf146, or shTnks and shTnks2 (n=8). (d) mRNA levels of cartilage-specific matrix genes in mouse chondrocytes treated with control siRNA, siRnf146, or siTnks and siTnks2 (n=5). (e, f) Protein levels of (e) cartilage-specific matrix genes or (0 SOX9 in mouse chondrocytes treated with control siRNA or siRnf146. (g) Factor loadings plot of 14 cartilage matrix genes in the cartilage of 16 BXD mouse strains in terms of transcript abundance, with Rnf146 added to the plot. (a, c, d) Data represent means±s.e.m. * P<0.05, ** P<0.01, *** P<0.001; ANOVA. (b, e, f).

FIGS. 5A to 5J are the results showing that Tankyrase inhibition enhances cartilage matrix gene expression in a SOX9-dependent manner, indicating that the synthesis of cartilage matrix is occurring through the regulation of Sox9 as shown above. (a-c) 4×48-p89 SOX9-dependent Col2a1 luciferase reporter assay in chondrocytes treated with (a) control shRNA, shTnks, shTnks2, or shTnks and shTnks2 (n=8), (b) XAV939, IWR-1, or PARP1/2 inhibitor ABT-888 (n≥5), or (c) 10 μM of various tankyrase inhibitors for 48 h (n=3). (d) 4×48-p89 SOX9-dependent Col2a1 luciferase reporter assay in chondrocytes transfected with control mock vector, wild-type TNKS2 vector, or PARP-dead (PD) TNKS2 mutant vector (n=3). (e) Box plot of fold changes of SOX9 target genes and other genes (two-tailed t test). SOX9 target genes are listed in Table 7. 4×48-p89 SOX9-dependent Col2a1 luciferase reporter assay in HEK293T cells transfected with mock vector or CMV-driven SOX9 expression vector and treated with (0 siTNKS and siTNKS2 (n=3) or (g) DMSO, XAV939, IWR-1, or ABT888 (n=4). (h) 4×48-p89 SOX9-dependent Col2a1 luciferase reporter assay in HEK293T cells expressing wild-type SOX9 or SOX9 with tankyrase-binding motif point mutation (n=6). SOX9 R2A mutant has both R257A and R271A mutations. (i) Knockdown efficiency of various Sox9 siRNAs in primary cultured mouse chondrocytes (n=3). siSox9 #2 was used throughout this study. (j) mRNA levels of cartilage-specific matrix genes in mouse chondrocytes transfected with control siRNA or siSox9 #2 followed by DMSO or XAV939 treatment for 72 h (n=6). (a-d, f-j) Data represent means±s.e.m. * P<0.05, ** P<0.01, *** P<0.001; ANOVA.

FIGS. 6A to 6G are the results showing Tankyrase inhibition ameliorates OA development in mice. (a, b) GSEA with OA-associated genesets in mouse chondrocytes treated with (a) siTnks and siTnks2 versus control siRNA (a) or tankyrase inhibitors versus vehicle (b). (c) Schematic illustration of the DMM model and drug treatment schedule. (d-g) Tankyrase inhibitors protect articular cartilage in surgically induced-OA mouse model. Cartilage destruction assessed by (d) Safranin O staining, (e) OARSI grade, and (f) immunostaining of cartilage matrix proteins or (g) SOX9. Scale Bar, (d) 500 μm, (f, g) 25 μm. (e) Data represent means±s.e.m. *** P<0.001; Kruskal-Wallis test.

FIGS. 7A to 7H are the results showing that Tankyrase inhibition stimulates chondrogenic differentiation of mesenchymal stem cells in vitro and in vivo. (a) Alcian Blue staining and absorbance quantitation of micromass cultured limb-bud mesenchymal cells treated with the indicated drugs (n=4). Scale bar: 1 mm (top), 300 μm (bottom). (b, c) Histology of hMSC pellets (b) treated with the indicated drugs or (c) infected with the indicated shRNA lentiviruses. Scale bars, 100 μm (top). (d) Knockdown efficiency of shTNKS and shTNKS2 in hMSC (right; n=4). (e-h) Tankyrase knockdown in mesenchymal stem cells regenerate articular cartilage in vivo. (e) Gross appearance (top) and histological images (middle and bottom) of cartilage lesions. ▾ indicates the graft sites. (f-h) Cartilage regeneration as evaluated using the (f) ICRS macroscopic score system (n=6), and (g) immunostaining of SOX9 and (h) cartilage matrix proteins in repair tissues in the defects. Scale bar, (g, h) 25 μm, (a, d, f) Data represent means±s.e.m. *P<0.05, **P<0.01, ***P<0.001; ANOVA (a, f) t test (d).

FIG. 8 is the result showing that cartilage matrix genes are not inter-correlated in non-cartilaginous organs. Heatmaps of Pearson correlation coefficients of transcript levels for cartilage matrix genes in bone femur, kidney, lung, and brain.

FIGS. 9A to 9D are the results showing that Tankyrase inhibition elicits cartilage-specific transcriptomic profile. (a) Volcano plots of gene expression changes in mouse chondrocytes treated with siTnks and siTnks2 or tankyrase inhibitors. Red dots represent genes with a fold-change of >3 and a FDR q of <1×10⁻⁵. Blue dots represent genes with a fold-change of <1/3 and a FDR q of <1×10⁻⁵. (b) Hierarchical clustering of fold changes of genes differentially expressed in chondrocytes in at least one condition (siTnks+siTnks2, XAV939, or IWR-1) compared to respective controls. RNA-Seq was conducted with three biological replicates (BR). (c) GO analysis on differentially expressed genes upregulated in all three conditions (siTnks+siTnks2, XAV939, and IWR-1). (d) Fold change heatmap of cartilage-signature genes in mouse chondrocytes treated with siTnks and siTnks2 or tankyrase inhibitors. List of cartilage-signature genes is provided in Table 8.

FIGS. 10A to 10D are the results showing that Tankyrase inhibition inverts gene expression profiles associated with OA cartilage. (a, b) Fold change heatmaps of OA-associated genes in mouse chondrocytes treated with siTnks and siTnks2 or tankyrase inhibitors. Genes that are upregulated and downregulated in OA cartilage are listed in Tables 9 and 10, respectively. (c) Heatmap of Pearson correlation coefficients between transcript levels of Tnks or Tnks2 and catabolic genes in the articular cartilage of 16 BXD mouse strains. (d) Correlation between Tnks or Tnks2 and catabolic regulators mRNA levels in the articular cartilage of 16 BXD mouse strains.

FIGS. 11A to 11F are the results showing that Tankyrase inhibition prevents progression of OA. (a) Light-emitting diode (LED) and fluorescence images of mouse knee joints intra-articularly injected with carrier-free DiD or DiD-loaded ascorbyl palmitate hydrogel. Images were acquired on the indicated days after injection. IR shows IR carrier-free immediate release and CR shows controlled release after hydrogel injection. (b) Experiments were done as in (a). Fluorescence images of mouse femur (femoral condyle) and tibia (tibial plateau) with carrier-free DiD or DiD-loaded ascorbyl palmitate hydrogel. Images were acquired at 9 days after IA injection (c) Immunostaining of MMP13 and β-catenin in articular cartilage of DMM-operated mouse. Scale bars: 50 μm. The percentage and the number of immunopositive cells are indicated. (d) Schematic representation of controlled drug delivery to DMM-operated mice started at 6 weeks after OA operation. (e, f) Cartilage destruction assessed by (e) Safranin O staining (scale bar: 200 μm) and (f) OARSI grade. Data represent means±s.e.m. *P<0.05; Mann-Whitney U test.

FIGS. 12A and 12B are the results showing that Tankyrase inhibition stimulates chondrogenic differentiation of mesenchymal stem cells in vivo. (a) hMSCs infected with control shRNA lentivirus or TNKS shRNA and TNKS2 shRNA lentiviruses were implanted in the full-thickness cartilage lesions of rat knee joints with fibrin gel constructs. A fibrin-only group was used as a control. Gross appearance of the indicated groups 8 weeks after transplantation. Transplantation of hMSCs with TNKS and TNKS2 knockdown resulted in superior healing, filling lesions with cartilage-like tissues. (b) Cartilage repair was assessed using various criteria of the ICRS visual histological score system for in vivo repaired cartilage (n=6). Data represent means±s.e.m. *P<0.05; ANOVA.

FIG. 13 is a schematic representation of the molecular mechanisms underlying the therapeutic effects of Tankyrase inhibitors in OA discovered herein.

### DETAILED DESCRIPTION OF THE EMBODIMENTS

The present disclosure is based on the discovery that TNKS (Tankyrase) functions as an upstream regulator of SOX9 which is known as an important player in the formation of cartilage matrix in chondrocytes. Specifically, in the present disclosure, it was identified that TNKS PARsylates SOX9, and the PARsylated SOX9 is then degraded through an intracellular protein degradation mechanism thus lowering the concentration of SOX9 in the cells. As a result, this makes it difficult for chondrocytes to synthesize cartilage specific matrix. In addition, it was identified herein the regulatory mechanism downstream of E3 Ubiquitin protein ligase involved in the degradation of SOX9. Furthermore, through the suppression of the mechanism using Tankyrase inhibitors that affect the mechanism identified herein, it was identified herein the effects of cartilage regeneration, arthritis treatment, and differentiation into chondrocytes.

Thus in one aspect of the present disclosure, there is provided a method of treating arthritis, or promoting cartilage regeneration or promoting differentiation of stem cells into chondrocytes. in a subject in need thereof comprising the step of administering to the subject an effective amount of an inhibitor of Tankyrase; or an modified adult stem cell in which the expression of Tankyrase is suppressed or a Tankyrase gene is knocked out, wherein the inhibitor of Tankyrase or the modified adult stem cell stabilizes the Sox9 protein or increases the concentration of the Sox9 protein by inhibiting the Tankyrase activity promoting the degradation of Sox9 protein.

Tankyrases (TNKS) is one of the 17 member of ARTD (Diphtheria toxin-like ADP-ribosyltransferase) enzyme superfamily (EC 2.4.2.30), and the ARTD is divided Polymerase (pARTD:ARTD1-6), and monotransferase (mARTD:ARTD7, 8, 10-12, 14-17) and inactive enzyme (ARTD9, 13) depending on the kind of amino acid present on the active site.

Human Tankyrase 1 (telomeric repeat binding factor 1 (TRF1)-inter-acting ankyrin-related ADP-ribose polymerase; TNKS1/ARTD5/PARP5a) and Tankyrase 2 (TNKS2/ARTD6/PARP5b) are multidomain protein having 1327 [NCBI DB: NP_003738.2] and 1166 [NCBI DB: NP_079511, AF329696.1] amino acids, respectively. Particularly, they have a catalytic domain at their C-terminal called ARTD responsible for ADP-ribosyltransferase activity. Human ARTD is also known as poly(ADP-ribose)polymerases (PARP), TNKS1 and TNKS2 have highly conserved sequences and 89% sequence identity. The conserved SAM domain is located N-terminal of ARTD domain and is involved in the formation of homo or hetero oligomers. Tankyrases also comprises ankyrin repeat consisting of five ankyrin repeat cluster involved in protein-protein interaction.

Particularly ARTD hydrolyze NAD+ (Nicotinamide adenine dinucleotide, oxidized form) into ADP-ribose (ADPr) and nicotinamide. After the hydrolysis, the nicotinamides are released from the binding site of ARTD and involved in the post-translational modification of proteins by attaching several ADP-ribose molecules to target proteins (Lehtio et al., Pharmacology of ADP ribosylation, Vol 280, pp 3576-3593).

In the present disclosure, it was identified that Tankyrase functions as an upstream regulator of SOX9. Tankyrase induces the degradation of SOX9 protein through PARsylation (poly(ADP-ribosyl)ation) thereof. In the meantime, SOX9 is known as a master transcription factor important in the formation of cartilage matrix such as collagen type 2 and Aggrecan in chondrocytes (Ng L J, Wheatley et al. Dev Biol. 1997; 183(1):108-21; Lefebvre V, et al. EMBO J. 1998; 17(19):5718-33; Wright E et al. Nat Genet. 1995; 9(1):15-20; Ohba S, et al. Cell Rep. 2015; 12(2):229-43).

Therefore, the control of SOX9 by regulating, particularly suppressing the upstream regulator Tankyrase can be utilized effectively for treating various disease or symptoms in which cartilage regeneration provides an effective treatment.

In one embodiment, the regeneration of cartilage is possible by promoting stem cells into chondrocytes. In one embodiment, using a mouse model with inducing degenerative arthritis, it was shown here that the injection of a tankyrase inhibitor through intraarticular promotes the regeneration of cartilage compared to a control group. Also, it was confirmed here that the stem cells with a genetic modification to suppress Tankyrase expression injected in a rat model with cartilage defects effectively are differentiated into chondrocytes regenerating cartilage.

In the present disclosure, the disease which requires a cartilage regeneration for effective treatment is osteoarthritis. Osteoarthritis is also commonly called degenerative arthritis. This is a disease in which the joint cartilage surrounding the joint surface of the bone is worn out exposing the bone under the cartilage, and the synovial membrane around the joint is inflamed, causing pain and deformation, and cartilage regeneration is essential for treatment.

In one embodiment, the Tankyrase inhibitor according to the present disclosure inhibits catalytic activity of the ARTD domains of TNKS1 and TNKS2. Therefore, as the Tankyrase inhibitor according to the present application, various inhibitors affecting the ADP-ribosyltransferase activity of ARTD or PARP can be used.

In one embodiment, the Tankyrase inhibitor is a substance that binds to a nicotinamide sub-region, adenosine sub-region, or both, which a sub-domain of the ARTD domain that is the catalytic region of the Tankyrase protein, or substance with tankyrase inhibitory function but the binding region of which is not identified. The sub-regions are known before (Lehtio et al., Pharmacology of ADP ribosylation, Vol 280, pp 3576-3593).

For example, the Tankyrase inhibitors that bind to nicotinamide sub-region are XAV939 {3,5,7,8-Tetrahydro-2-[4-(trifluoromethyl)phenyl]-4H-thiopyrano[4,3-d]pyrimidin-4-one} or MN-64 (2-[4-(1-Methylethyl)phenyl]-4H-1-benzopyran-4-one); the adenosine sub-region binding inhibitors are IWR-1 [4-(1,3,3a,4,7,7a-Hexahydro-1,3-dioxo-4,7-methano-2H-isoindol-2-yl)-N-8-quinolinyl-Benzamide], JW55 {N-[4-[[[[Tetrahydro-4-(4-methoxyphenyl)-2H-pyran-4-yl]methyl]amino]carbonyl]phenyl]-2-furancarboxamide}, WIKI4 2-[3-[[4-(4-Methoxyphenyl)-5-(4-pyridinyl)-4H-1,2,4-triazol-3-yl]thio]propyl]-1Hbenz[de]isoquinoline-1,3(2H)-dione, TC-E5001 (3-(4-Methoxyphenyl)-5-[[[4-(4-methoxyphenyl)-5-methyl-4H-1,2,4-triazol-3-yl]thio]methyl]-1,2,4-oxadiazole) or G007-LK [(E)-4-(5-(2-(4-(2-chlorophenyl)-5-(5-(methylsulfonyl)pyridin-2-yl)-4H-1,2,4-triazol-3-yl)vinyl)-1,3,4-oxadiazol-2-yl)benzonitrile]; and the inhibitors with unidentified binding region are G244-LM {3,5,7,8-tetrahydro-2-[4-[2-(methylsulfonyl)phenyl]-1-piperazinyl]-4H-thiopyrano[4,3-d]pyrimidin-4-one}, or AZ6102 {rel-2-[4-[6-[(3R,5S)-3,5-Dimethyl-1-piperazinyl]-4-methyl-3-pyridinyl]phenyl]-3,7-dihydro-7-methyl-4H-pyrrolo[2,3-d]pyrimidin-4-one}, or isomers or derivatives thereof are included herein, without being limited thereto. The skilled person in the art would be able to select appropriate inhibitors or isomers or derivatives thereof considering what is disclosed herein.

In other embodiment, Tankyrase inhibitors which may be employed herein are siRNA (small interfering RNA) or shRNA (small hairpin RNA) or miRNA (microRNA). The siRNA, shRNA and miRNA are silencing mRNA transcripts through RNA interference by forming RISC (RNA Induced Silencing Complex) in which siRNAs sequence specifically bind to the mRNA transcripts. siRNA, shRNA and miRNA have a sequence significantly complementary to their target sequence. The term significant complementarity means a sequence having at least about 70%, about 80%, about 90%, or about 100% complementary to at least 15 consecutive bases of a target sequence. Various antisense oligonucleotides, siRNA, shRNA and/or miRNA targeting Tankyrase from various sources may be used for the present disclosure as long as they bind to a target sequence to silence them. Also biological equivalent, derivatives and analogues thereof are also included. Antisense oligonucleotides is a short synthetic nucleotides known in the art, and they bind to a coding sequence of a target protein and suppress/decrease the expression level of a target protein. Antisense RNA may have an optimum length according to the methods of transfer or types of target genes and be for example 6, 8 or 10 to 40, 60 or 100 bases in length. In one embodiment, siRNA is used to suppress the expression of Tankyrase gene. In one embodiment, sequences of such siRNAs are represented by SEQ ID Nos: 25 and 26 for sense and antisense, respectively for TNKS1, and SEQ ID Nos: 27 and 28 for sense and antisense, respectively for TNKS2.

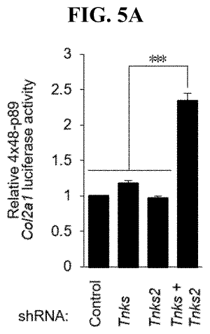

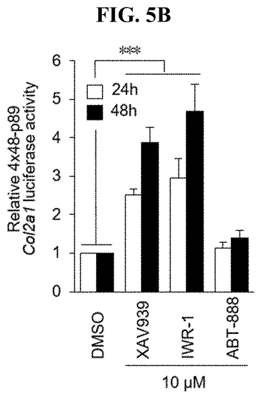

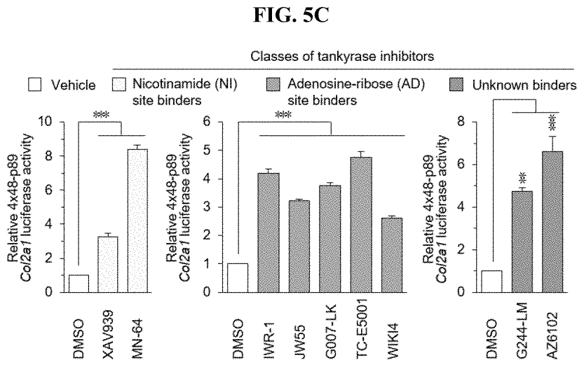

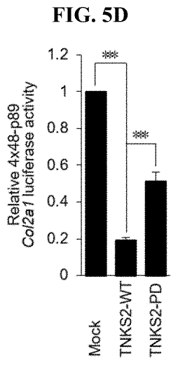

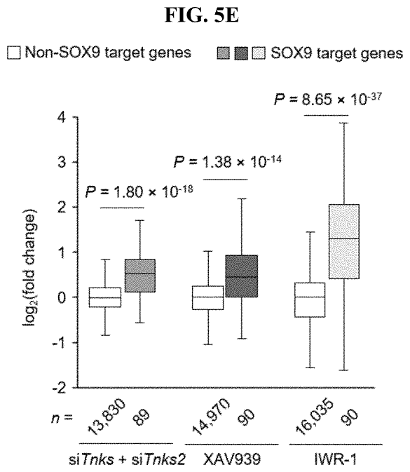

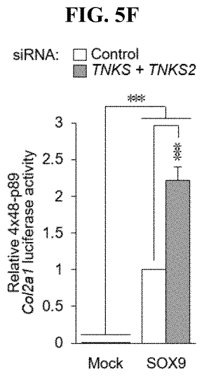

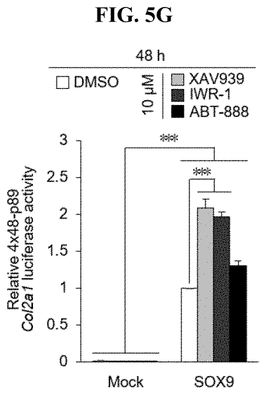

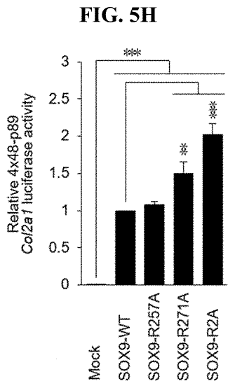

The above sequences may be used as dsRNA in which a sense and antisense sequences bind to each other. Further such sequences may further comprise at its 3′ terminal dTdT overhang. As described in FIG. 5, such siRNAs effectively suppress the expression of TNKS1/2 at the cellular level and thus increasing the concentration of SOX9. This indicates that siRNAs can be effectively used for the treatment of cartilage regeneration and arthritis.

As used herein, the terms “treat,” “treatment,” and “treating” include alleviating, abating or ameliorating at least one symptom of a disease or condition, and/or reducing severity, progression and/or duration thereof, and/or preventing additional symptoms, and includes prophylactic and/or therapeutic measures. The disease or symptoms includes disease or symptoms that requires cartilage regeneration for effective treatment.

The terms “individual,” “subject,” and “patient,” are used interchangeably herein and refer to any mammalian subject for whom diagnosis, treatment, or therapy is desired, particularly humans.

The present composition may further include one or more pharmaceutically acceptable carriers, which includes but does not limited to, saline, sterilized water, Ringer's solution, buffered saline, dextrose solution, maltodextrin solution, glycerol, ethanol, liposome. If desired, the composition may further include antioxidant, buffer, antibacterial agents, and other additives known in the art to prepare pharmaceutical compositions. The present composition may be formulated into injectable formulations or oral formulations such as capsules, granules, or tablets by methods known in the art using one or more of diluents, dispersing agents, surfactants, binders and lubricants. Also encompassed for the present invention is a target specific composition combined with an antibody or other ligands that specifically recognize a molecule present on a target tissue or organ of interest. Further latest edition of Remington's Pharmaceutical Science (Mack Publishing Company, Easton Pa.) may be referred for the preparation and formulation of pharmaceutical composition.

The present composition can be administered by various routes known in the art such as oral or parenteral delivery for example intravenous, subcutaneous, or intraperitoneal injections or delivery through patch, nasal or respiratory patches. In one embodiment, injections are preferred. Desirable or optimal dosage may vary among patients depending on various factors such as body weight, age, sex, general condition of health, diet, severity of diseases, and excretion rate. Dosages used for known TNKS inhibitors may be referred. Where siRNA, miRNA, antisense oligonucleotides, shRNA are used, parenteral deliveries are preferred. The typical unit dosage includes but does not limit to for example about 0.01 mg to 100 mg a day. Typical daily dosage ranges from about 1 μg to 10 g and may be administered one or multiple times a day.

In other aspect, the present disclosure relates to a composition or cell therapy agent for treating arthritis comprising stem cells genetically modified to suppress the expression of Tankyrase.

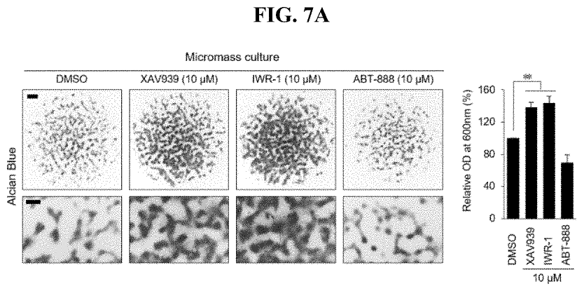

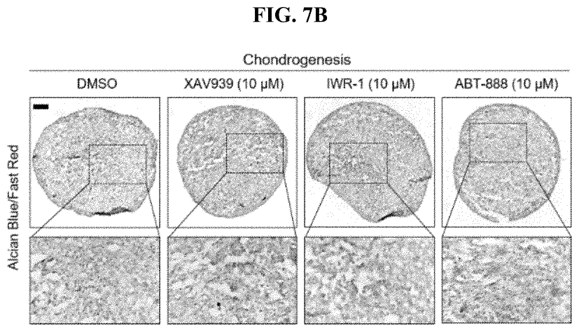

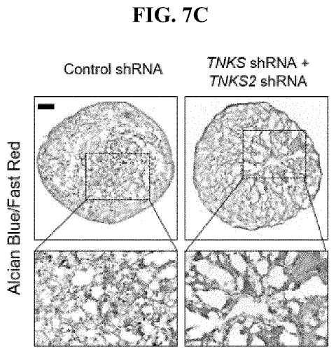

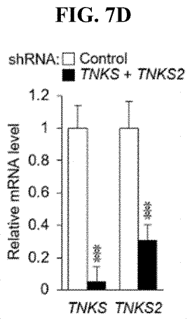

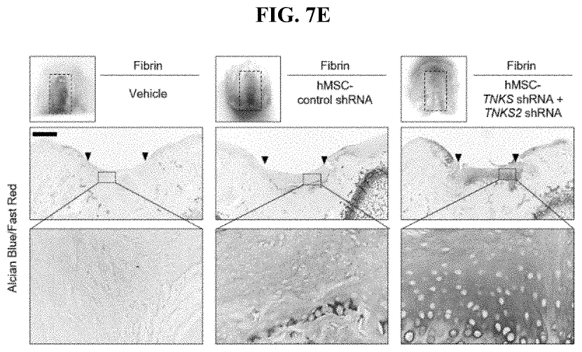

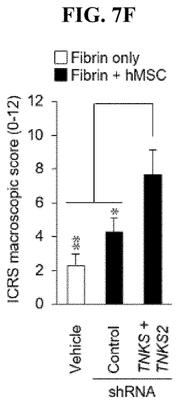

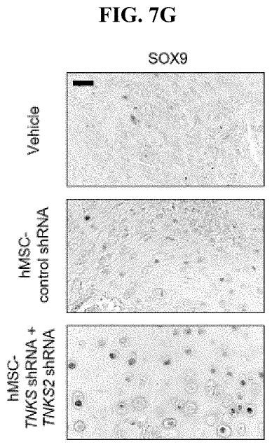

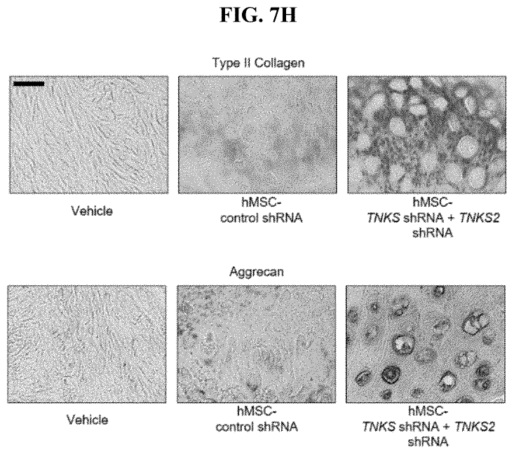

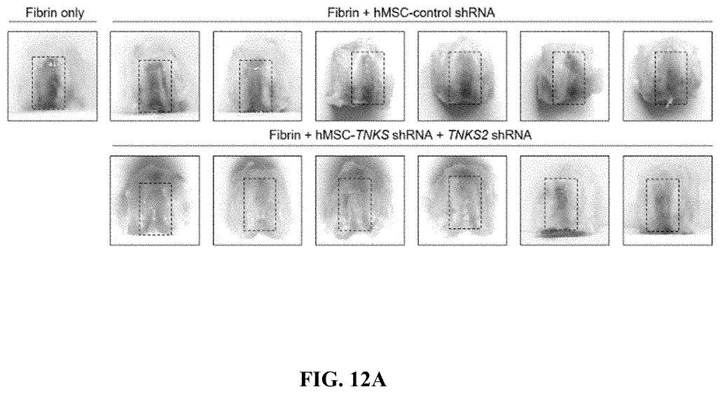

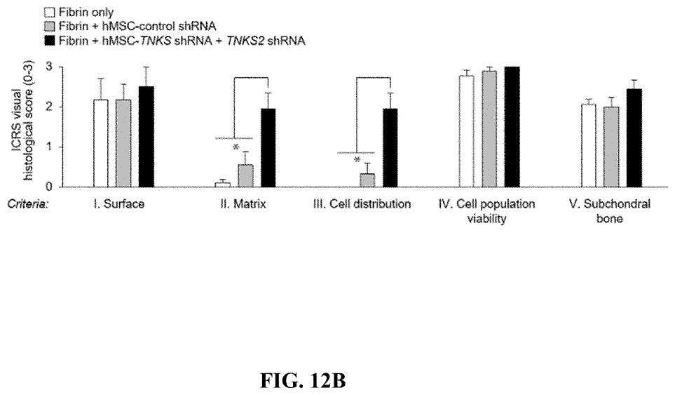

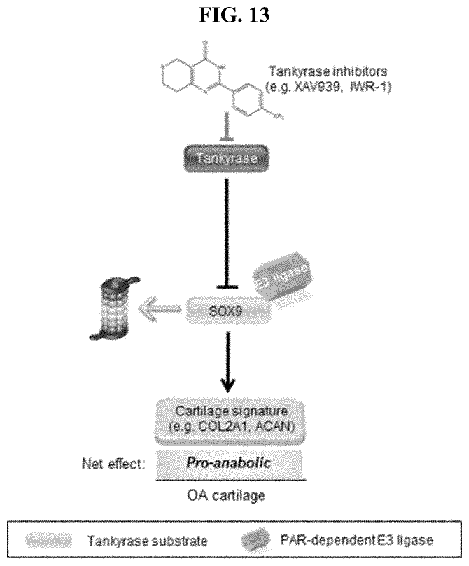

As disclosed in FIGS. 7 and 12, the stem cells modified to suppress the expression of Tankyrase and the stem cells in which Tankyrases genes are knocked out is able to treat arthritis.

In the present disclosure, the suppression of Tankyrase includes the suppression of the transcription of Tankyrase genes into mRNAs or translation of Tankyrase mRNA into proteins or both.

In one embodiment, the suppression may be accomplished by using shRNA specific to Tankyrase. In other embodiment, Tankyrase gene may be knocked out. A skilled person in the art would be able to select appropriate methods to suppress the expression of Tankyrase in stem cells in consideration of the conventional knowledge in the art and what is disclosed herein.

In the present disclosure, the term “Mesenchymal Stem Cell (MSC)” refers to an adult stem cell which is a pluripotent or multipotent cell obtained from various part of an adult body such as cord blood, bone marrow, blood, dermis, or periosteum. It can differentiate into cartilage cells. The mesenchymal stem cell may be from an animal, preferably a mammal, more preferably a human mesenchymal stem cell. More particularly, it may be stem cells present in cartilage.

The process of obtaining mesenchymal stem cells is known in the art. The mesenchymal stem cells are isolated from a human or mammalian, preferably human mesenchymal stem cell source. Then the isolated cells are incubated in an appropriate medium. During the culture, the suspended cells are removed and the cells attached to the culture plate are passaged to obtain finally established mesenchymal stem cells. The mesenchymal stem cells can be identified, for example, through flow cytometry.

The composition of the present disclosure may be referred to a cell therapeutic agent. The term cell therapeutic agents refer to a medicine which is prepared by modifying the cells of autologous, allogenic or xenogeneic origin in vitro using biological, chemical or physical methods such as proliferation or selection, in which the cells are used as a therapeutic agent to replace or repair defect cells in the body. The cell therapeutic agents are controlled as a medicine in US from 1993 and in Korea from 2002.

The MSC which may be comprised in the present cell therapeutic agent may be of autologous, allogenic or xenogeneic origin. More preferably, it is autologous.

In one embodiment, MSC which may be comprised in the present cell therapeutic agent is from animal, preferably mammals, more preferably from human beings.

The route of administration of a cell therapeutic agent or a pharmaceutical composition comprising cells according to the present application can be administered through any general route as long as it can reach the target tissue. Parenteral administration may be, for example, intraperitoneal administration, intravenous administration, intramuscular administration, subcutaneous administration, intradermal administration, but is not limited thereto. In one embodiment, the composition according to the present invention may be administered in a manner that is intravenously administered or injected directly into an organ in need of administration of a cell or composition according to the present invention.

The present composition may be formulated with pharmaceutically acceptable carriers. Pharmaceutically acceptable carriers include, for example, carriers for parenteral administration such as water, suitable oils, saline, aqueous glucose and glycols, and may further include stabilizers and preservatives. Suitable stabilizers include antioxidants such as sodium hydrogen sulfite, sodium sulfite or ascorbic acid. Suitable stabilizers include antioxidants such as sodium hydrogen sulfite, sodium sulfite or ascorbic acid. Suitable preservatives include benzalkonium chloride, methyl- or propyl-parabens and chlorobutanol. In addition, the composition for cell therapy according to the present invention, if necessary, depending on the method of administration or formulation, suspending agent, solubilizing agent, stabilizer, isotonic agent, preservative, anti-adsorption agent, surfactant, diluent, excipient, pH adjuster, painless agent, buffers, antioxidants, and the like. Pharmaceutically acceptable carriers and formulations suitable for the present invention, including those exemplified above, are described in detail in Remington's Pharmaceutical Sciences, latest edition.

The composition for cell therapy of the present invention is formulated in a unit dose form by formulating using a pharmaceutically acceptable carrier and/or excipient according to a method that can be easily carried out by a person skilled in the art to which the present invention pertains. The composition may also be administered by any device capable of transporting the cell therapy agent to the target cell. The cell therapy composition of the present invention may include a therapeutically effective amount of a cell therapy agent for the treatment of a disease.

As used herein, the term “therapeutically effective amount” or “effective amount” refers to the amount of a therapy, which is sufficient to treat, attenuate, reduce the severity of arthritis such as osteoarthritis, reduce the duration of arthritis such as osteoarthritis, prevent the advancement of arthritis such as osteoarthritis, cause regression of arthritis such as osteoarthritis, ameliorate one or more symptoms associated with arthritis such as osteoarthritis, or enhance or improve the therapeutic effect(s) of another therapy. The exact amount of TNKS inhibitor or cell therapeutic agents may vary depending the desired effects.

The optimal amount can be readily determined by one of skill in the art, including the type of disease, the severity of the disease, the content of other ingredients in the composition, the type of formulation, and the patient's age, weight, general health status, sex and diet, It can be adjusted according to various factors including the time of administration, route of administration and secretion rate of the composition, duration of treatment, and drugs used simultaneously.

In one embodiment according to the present application, the cell therapy agent may be administered in the knee joint cavity.

It is important to consider all of the above factors and include an amount that can achieve the maximum effect in a minimal amount without side effects. For example, the dosage of the composition of the present invention may be 1.0×10⁷ to 1.0×10⁸ cells/kg (body weight), more preferably 1.0×10⁵ to 1.0×10⁸ cells/kg (body weight) based on the active ingredient. However, the dosage may be variously prescribed by factors such as the formulation method, the administration method, the patient's age, weight, sex, food, administration time, administration route, excretion rate, and response sensitivity, and those skilled in the art taking these factors into consideration, the dosage can be appropriately adjusted. The number of times of administration may be one or two or more times within the range of clinically acceptable side effects, and the administration site may be administered at one site or two or more sites.

The present disclosure is further explained in more detail with reference to the following examples. These examples, however, should not be interpreted as limiting the scope of the present invention in any manner.

### EXAMPLES

Methods

In silico analysis of multi-tissue transcriptomes of the BXD mouse population. Cartilage (GN208) (Suwanwela, J. et al. Systems genetics analysis of mouse chondrocyte differentiation. J Bone Miner Res 26, 747-760, doi:10.1002/jbmr.271 (2011), bone femur (GN411) (Zhu, M. et al. Activation of beta-catenin signaling in articular chondrocytes leads to osteoarthritis-like phenotype in adult beta-catenin conditional activation mice. J Bone Miner Res 24, 12-21, doi:10.1359/jbmr.080901 (2009)), kidney (GN118), lung (GN160) Alberts, R., Lu, L., Williams, R. W. & Schughart, K. Genome-wide analysis of the mouse lung transcriptome reveals novel molecular gene interaction networks and cell-specific expression signatures. Respir Res 12, 61, doi:10.1186/1465-9921-12-61 (2011), and brain (GN123)(Saba, L. et al. Candidate genes and their regulatory elements: alcohol preference and tolerance. Mamm Genome 17, 669-688, doi:10.1007/s00335-005-0190-0 (2006)) data sets were obtained from GeneNetwork (www.genenetwork.org). illuminaMousev1.db 1.26.0 (http://bioconductor.org/packages/illuminaMousev1.db/) and mouse4302. db 3.2.3 R. (http://bioconductor.org/packages/illuminaMousev1p1.db/) were used for probe reannotation. For the cartilage and bone femur data sets, a probe that did not overlap with any known SNPs, perfectly and uniquely matched the target transcript, and also had the highest expression was used for each transcript. For other data sets, probes having the highest expression were used for each transcript. Data sets were clustered using a hierarchically clustered algorithm (complete connection and correlation deviated from the center) at cluster 3.0 (http://bonsai.hgc.jp/˜mdehoon/software/cluster/software.htm) and correlation heatmap was drawn using Perez-Llamas, C. & Lopez-Bigas, N. Gitools: analysis and visualization of genomic data using interactive heat-maps (PLoS One 6, e19541, doi:10.1371/journal.pone.0019541 (2011) Gitools 2.3.1. IBM SPSS Statistics 24 (http://www.ibm.com/analytics/us/ko/technology/spss/). Major components analysis was used to obtain two factors, and the factor points were calculated using Regression method.

Primary culture of mouse articular chondrocytes. For the primary culture of mouse articular chondrocytes, cells were isolated from femoral condyles and tibial plateaus of 4-5-day-old ICR mice, as described previously⁸³. Chondrocytes were maintained in DMEM supplemented with 10% fetal bovine serum (FBS), 100 units/ml penicillin, and 100 μg/ml streptomycin, and cells were treated as indicated in each experiment. Transfection was performed with METAFECTENE PRO (Biontex) according to the manufacturer's protocol. Small interfering RNAs (siRNAs) used for RNA interference (RNAi) in mouse articular chondrocytes are listed in Table 1. All siRNAs, including negative control siRNA, were purchased from Bioneer. Recombinant mouse Wnt-3a (315-20) was purchased from PeproTech, and recombinant mouse Dkk-1 (5897-DK) was purchased from R&D Systems.

RT-PCR and qPCR. Total RNAs were extracted using TRI reagent (Molecular Research Center, Inc.). RNAs were reverse transcribed using EasyScript Reverse Transcriptase (Transgen Biotech). Then, cDNA was amplified by PCR or qPCR with the primers listed in Table 2. qPCR was performed with SYBR TOPreal qPCR 2× preMIX (Enzynomics) to determine transcript abundance. Transcript quantity was calculated using the ΔΔCₜ method, and Hprt or HPRT1 levels were used as housekeeping controls. The log 2 (fold change) value of the cartilage stromal gene of mouse articular chondrocytes treated with siRNA was clustered using a hierarchical clustering algorithm (mean association and central correlation distance) in 1.0.4 R package. PCA was performed using the same R package.

Whole-cell lysate preparation. Whole-cell lysates were prepared in RIPA buffer (150 mM NaCl, 1% NP-40, 50 mM Tris, pH 8.0, 0.5% sodium deoxycholate, 0.1% SDS) supplemented with a protease inhibitor cocktail (Sigma-Aldrich).

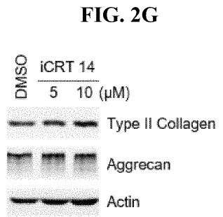

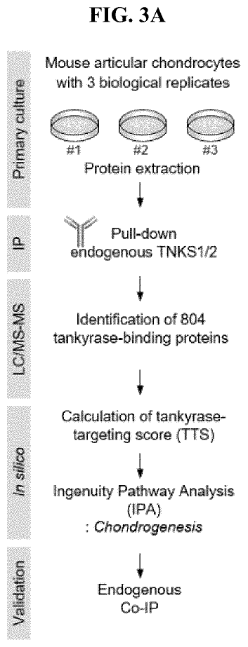

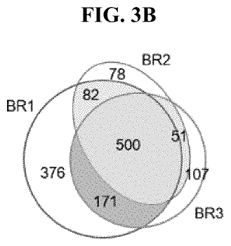

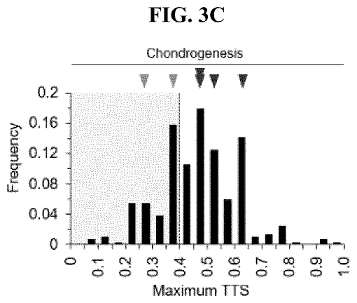

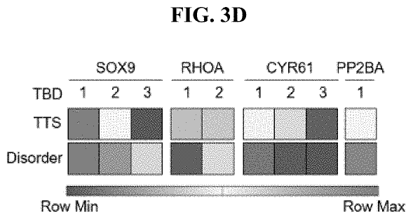

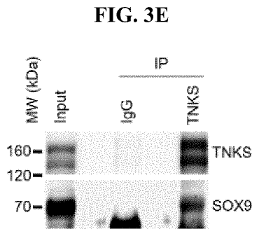

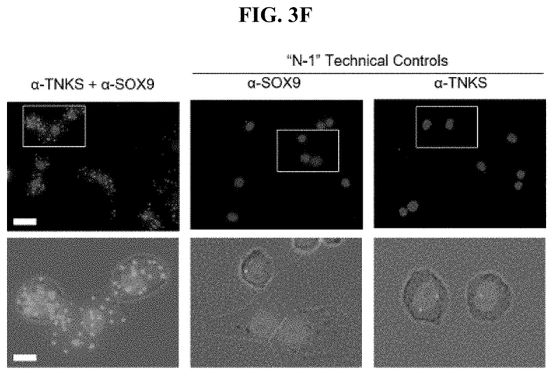

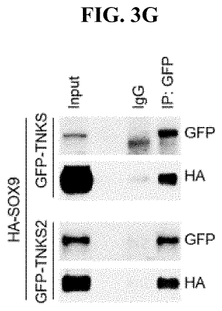

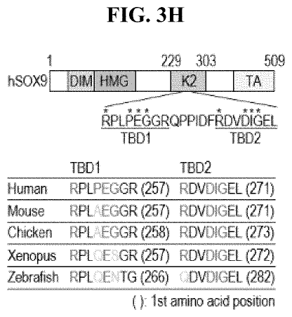

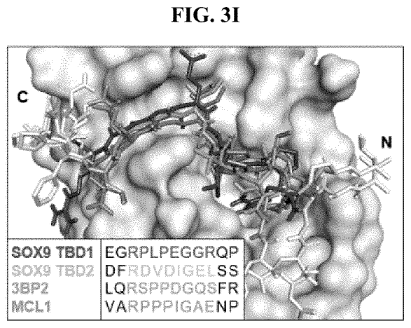

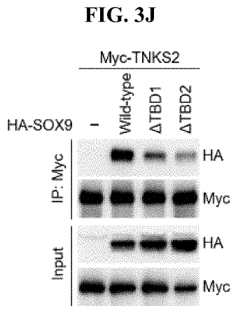

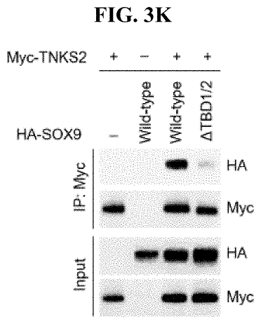

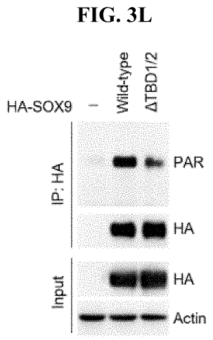

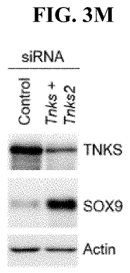

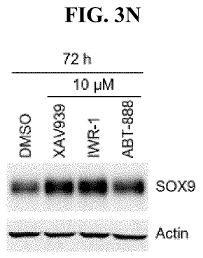

Antibodies. Anti-FLAG tag antibody (3165) was purchased from Sigma-Aldrich. Antibodies against GFP (sc-9996), Sox-9 (sc-20095), Sox-9 (sc-166505), Tankyrase-1/2 (sc-8337), Tankyrase-1/2 (sc-365897), Ubiquitin (sc-8017), and Actin (sc-1615), normal Mouse IgG(sc-2025), normal rabbit IgG(sc-2027) were purchased from Santa Cruz Biotechnology. Sox-9 (sc-20095) antibody was used only in FIG. 3e, m and Sox-9 (sc-166505) antibody was used only in FIG. 3g. Antibodies against aggrecan (AB1031), type II collagen (MAB8887), and human mitochondria (MAB1273) were purchased from Millipore, and antibodies against Myc tag (2276) and Sox9 (82630) were purchased from Cell Signaling Technology. Prior to detection of aggrecan, samples were treated with chondroitinase ABC (C3667) from Sigma-Aldrich. Anti-β-Catenin antibody (610154) was obtained from BD Biosciences. Anti-Poly(ADP-ribose) antibody (AG-20T-0001) was purchased from AdipoGen. All primary antibodies were used according to the manufacturer's protocol.

Transcript inhibitors of Tankyrase, PARP1/2 and β-catenin response. XAV939 (X3004), IWR-1 (I0161), JW55 (SML0630), and WIKI4 (SML0760) were obtained from Sigma-Aldrich. G007-LK (B5830) were purchased from Apexbio, G244-LM (1563007-08-8) was from AOBIOUS, MN-64 (HY19351) from MedChem Express, AZ6102 (S7767) from SelleckChem, and TC-E 5001 (5049) from Tocris. Tankyrase inhibitors were classified into three different classes depending on their mode of action (Lehtio, L., Chi, N. W. & Krauss, S. Tankyrases as drug targets. FEBS J 280, 3576-3593, doi:10.1111/febs.12320 (2013) [symbol] Haikarainen, T., Krauss, S. & Lehtio, L. Tankyrases: structure, function and therapeutic implications in cancer. Curr Pharm Des 20, 6472-6488 (2014)). ABT-888 (11505) was purchased from Cayman, and iCRT 14 (4299) from Tocris.

RNA sequencing (RNA-seq). Primary cultured mouse articular chondrocytes were treated with DMSO or 10 μM of XAV939 or IWR-1 for 108 h or transfected with control siRNA or Tnks and Tnks2 siRNAs. Three biological replicates were used for each group. One microgram of high-quality RNA samples (RIN>7.0) were used to construct RNA-seq libraries with the TruSeq Stranded mRNA Library Prep kit (Illumina). Libraries were validated with an Agilent 2100 Bioanalyzer. RNA-seq was performed on an Illumina HiSeq 2500 sequencer at Macrogen. The sequence reads were trimmed with Trimmomatic⁸⁶ and mapped against the mouse reference genome (mm10) using TopHat. Read counts per gene were calculated using HTSeq⁸⁸. Differential expression analysis was conducted using the DESeq2 R package⁸⁹. DEGs were selected using a |fold change| cutoff of >3 and a FDR q cutoff of <1×10⁻⁵. DEGs at least one condition were clustered with hierarchical clustering algorithm (ward.D linkage with euclidean distance) using gplots R package. GO analysis was conducted using Enrichr⁹⁰. Heatmaps of DEGs that are in the cartilage-signature gene set or the osteoarthritis-signature gene sets were drawn with Gitools.

GSEA analysis. Genes were ranked according to the shrunken log₂ fold change calculated via DESeq2. GSEA (Subramanian, A. et al. Gene set enrichment analysis: a knowledge-based approach for interpreting genome-wide expression profiles. Proc Natl Acad Sci USA 102, 15545-15550, doi:10.1073/pnas.0506580102 (2005)) was performed in pre-ranked mode, with all default parameters, for the cartilage-signature gene set or the osteoarthritis-signature gene sets. A ten-thousand permutations were used to calculate P values.

Generation of a cartilage-signature gene set. Microarray data for nasal chondrocytes at embryonic day 17.5 and rib chondrocytes at postnatal day 1 were obtained from GSE69108 (Ohba, S., He, X., Hojo, H. & McMahon, A. P. Distinct Transcriptional Programs Underlie Sox9 Regulation of the Mammalian Chondrocyte. Cell Rep 12, 229-243, doi:10.1016/j.celrep.2015.06.013 (2015)). Microarray data for mouse embryonic fibroblasts (MEFs) were obtained from GSM577694, GSM577695, and GSM577696 of GSE23547(Brellier, F. et al. Tenascin-C triggers fibrin accumulation by downregulation of tissue plasminogen activator. FEBS Lett 585, 913-920, doi:10.1016/j.febslet.2011.02.023 (2011)). The limma R package (Ritchie, M. E. et al. limma powers differential expression analyses for RNA-sequencing and microarray studies. Nucleic Acids Res 43, e47, doi:10.1093/nar/gkv007 (2015)) was used to compute differential expression between nasal chondrocytes and MEFs or between rib chondrocytes and MEFs. The probe with the highest expression was used for each transcript. Genes with a fold-change of >5 and a FDR q of <1×10⁻⁵ in both nasal chondrocytes and rib chondrocytes compared to MEFs were selected as cartilage-signature genes. The cartilage-signature genes are listed in Table 8.

Immunoprecipitation. Except for FIG. 3f, the cells were pretreated with 10 μM MG-132 (A2585) from ApexBio for 6 hrs. Cell lysates were prepared using EBC200 buffer (50 mM Tris-HCl, pH 7.4, 150 mM NaCl, 0.5% NP-40 and 1 mM EDTA) supplemented with the protease inhibitor cocktail. Cell lysates were used for pulldown with the indicated antibodies and protein A/G-Sepharose beads (GE Healthcare). For detection of PARylated proteins, 5 μM of ADP-HPD (118415) from Calbiochem was added to the lysis buffer. For ubiquitin analysis, 100 mM N-ethylmaleimide (E3876) from Sigma-Aldrich was added to the lysis buffer. The mixtures were incubated at 4° C. overnight and washed 5 times EBC200. The bound proteins were subjected to SDS-PAGE or LC-MS/MS analysis.

Endogenous tankyrase1/2 pulldown and mass spectrometry. Primary cultured mouse articular chondrocytes were grown for 4 days and treated with 10 μM MG132 (Apexbio, A2585). Cells were lysed, and lysates were incubated with normal rabbit IgG or anti-tankyrase antibody. The bound proteins were eluted with 8 M urea in 50 mM NH₄HCO₃ buffer, pH 8.2 for 1 h at 37° C., and in-solution digestion was performed as described previously (Kim, J. S., Monroe, M. E., Camp, D. G., 2nd, Smith, R. D. & Qian, W. J. In-source fragmentation and the sources of partially tryptic peptides in shotgun proteomics. J Proteome Res 12, 910-916, doi:10.1021/pr300955f (2013)). Peptide sequencing was carried out by LC-MS/MS on a Thermo Ultimate 3000 RSLCnano high-pressure liquid chromatography system coupled to a Thermo Q-Exactive Hybrid Quadrupole-Orbitrap mass spectrometer. LC-MS/MS raw data were converted into .mzML files using ProteoWizard MSConvert (Chambers, M. C. et al. A cross-platform toolkit for mass spectrometry and proteomics. Nat Biotechnol 30, 918-920, doi:10.1038/nbt.2377 (2012)), and the MS-GF+ algorithm (Kim, S. & Pevzner, P. A. MS-GF+ makes progress towards a universal database search tool for proteomics. Nat Commun 5, 5277, doi:10.1038/ncomms6277 (2014)) with a parameter file consisting of no enzyme criteria and static cysteine modification (+57.022 Da) was used for comparison of all MS/MS spectra against the mouse Uniprot database. The final peptide identifications had <1% false discovery rate (FDR) q, at the unique peptide level. Only fully tryptic and semitryptic peptides were considered. For each biological replicate, proteins that were detected only once and proteins that were coimmunoprecipitated with normal rabbit IgG were not considered. For proteins detected in more than one biological replicate, the peptides and proteins are listed in Table 10. The Venn diagram was drawn with eulerAPE (Micallef, L. & Rodgers, P. eulerAPE: drawing area-proportional 3-Venn diagrams using ellipses. PLoS One 9, e101717, doi:10.1371/journal.pone.0101717 (2014)).

In silico prediction of tankyrase substrate proteins. The 8×20 position-specific scoring matrix (PSSM) generated in Guettler et al (Structural basis and sequence rules for substrate recognition by Tankyrase explain the basis for cherubism disease. Cell 147, 1340-1354, doi:10.1016/j.cell.2011.10.046 (2011)) was used to calculate a TTS for each octapeptide in the proteins identified by LC-MS/MS.

$$TTS = \frac{{\sum }_{pos . = 0}^{8} {PSSM}_{pos .}}{max ( {\sum }_{pos . = 0}^{8} {PSSM}_{pos .} )}$$

The Python code for calculating the maximum TTS for each tankyrase binding protein is in the Supplementary Source Code. Only those proteins having at least one octapeptide with a TTS of ≥0.385 were considered. This cutoff is the TTS of the tankyrase-binding motifs of mouse AXIN1 and AXIN2. AXIN1 and AXIN2, known tankyrase substrates (Huang, S. M. et al. Tankyrase inhibition stabilizes axin and antagonizes Wnt signalling. Nature 461, 614-620, doi:10.1038/nature08356 (2009)), have the lowest maximum TTS among the known tankyrase substrates, due to the suboptimal amino acids at the 4^{th} and 5^{th} positions (Guettler, S. et al. Structural basis and sequence rules for substrate recognition by Tankyrase explain the basis for cherubism disease. Cell 147, 1340-1354, doi:10.1016/j.cell.2011.10.046 (2011). For further screening, the chondrogenesis category in IPA (https://www.qiagenbioinformatics.com/products/ingenuity-pathway-analysis/) was used. The mouse proteins in the IPA chondrogenesis category are listed in Table 6. For the candidate proteins, IUPred disorder scores were calculated for the octapeptides with a TTS of ≥0.385. The heatmap of TTS and IUPred disorder scores for candidate proteins was drawn with Gitools 2.3.1 (Perez-Llamas, C. & Lopez-Bigas, N. Gitools: analysis and visualisation of genomic data using interactive heat-maps. PLoS One 6, e19541, doi:10.1371/journal.pone.0019541 (2011)).

Cell line culture. HEK293 and HEK293T cells were cultured in DMEM containing 10% FBS, 100 units/ml penicillin, and 100 μg/ml streptomycin. Transfection was performed with METAFECTENE PRO (Biontex) or PEI transfection reagent (Sigma-Aldrich) according to the manufacturer's protocol. The siRNAs used in HEK293T are listed in Table 1. The siRNA sequences targeting TNKS or TNKS2 were described previously (Huang, S. M. et al. Tankyrase inhibition stabilizes axin and antagonizes Wnt signalling. Nature 461, 614-620, doi:10.1038/nature08356 (2009)).

Plasmids. Human SOX9 cDNA (hMU008919) was purchased from Korea Human Gene Bank and subcloned into a pcDNA3-HA plasmid or a p3×FLAG-CMV10 plasmid (see Table 3 for PCR primers used for subcloning). To express human SOX9 under TK promoter, Renilla luciferase gene in a pRL-TK plasmid was replaced by 3×FLAG-SOX9. To generate mutant constructs, PCR-mediated mutagenesis was conducted (see Table 4 list of PCR primers used). The GFP-tagged human TNKS plasmid was a gift from Dr. Chang-Woo Lee, and the Myc-tagged human TNKS2 plasmid was a gift from Dr. Junjie Chen. The FLAG-tagged human TNKS2 plasmid and the FLAG-tagged human TNKS2 M1054V plasmid were gifts from Dr. Nai-Wen Chi (Sbodio, J. I., Lodish, H. F. & Chi, N. W. Tankyrase-2 oligomerizes with tankyrase-1 and binds to both TRF1 (telomere-repeat-binding factor 1) and IRAP (insulin-responsive aminopeptidase). Biochem J 361, 451-459 (2002)). The 4×48-p89 SOX9-dependent Col2a1 luciferase reporter construct was a gift from Dr. Veronique Lefebvre (Murakami, S., Lefebvre, V. & de Crombrugghe, B. Potent inhibition of the master chondrogenic factor Sox9 gene by interleukin-1 and tumor necrosis factor-alpha. J Biol Chem 275, 3687-3692 (2000)). Human TNKS2 cDNA was subcloned into a pEGFP-C1 plasmid to construct a GFP-tagged human TNKS2 plasmid (see Table 3 for primers used for subcloning). A control shRNA sequence was inserted into the pLKO.1 puro and pLKO.1 hygro plasmids. Human TNKS and TNKS2 shRNA sequences were inserted into the pLKO.1 puro and pLKO.1 hygro plasmids, respectively. The shRNA sequences targeting human TNKS or TNKS2 were as described previously (Huang, S. M. et al. Tankyrase inhibition stabilizes axin and antagonizes Wnt signalling. Nature 461, 614-620, doi:10.1038/nature08356 (2009)). Mouse Tnks and Tnks2 shRNA sequences were inserted into the pLKO.1 puro and pLKO.1 hygro plasmids, respectively. Mouse Rnf146 shRNA sequence was inserted into the pLKO.1 puro plasmid. The shRNA sequence targeting mouse Tnks was as described previously (Levaot, N. et al. Loss of Tankyrase-mediated destruction of 3BP2 is the underlying pathogenic mechanism of cherubism. Cell 147, 1324-1339, doi:10.1016/j.cell.2011.10.045 (2011)). The primers used to generate the above plasmids are listed in Tables 3,4 and 5.

in situ PLA. Primary cultured mouse articular chondrocytes were used for in situ PLA. Duolink® PLA was performed according to the manufacturer's protocol (Sigma-Aldrich). Antibodies against Sox-9 (sc-166505) and Tankyrase-1/2 (sc-8337) were used to recognize endogenous mouse SOX9 and endogenous mouse tankyrase, respectively.

Sequence alignment of TBD1 and TBD2 of SOX9 among vertebrates. For the sequence alignment of TBD1 and TBD2 of SOX9 among vertebrates, NP_000337.1 (Homo sapiens SOX9), NP_035578.3 (Mus musculus SOX9), NP_989612.1 (Gallus SOX9), NP_001016853.1 (Xenopus tropicalis SOX9), and NP_571718.1 (Danio rerio SOX9) were used.

Structural modeling of protein-peptide interactions. GalaxyPepDock¹⁰⁰ was used for modeling of the ARC4 domain of human TNKS2 in complex with the TBD1 or TBD2 peptide of human SOX9. The structures of ARC4:3BP2 (PDB ID: 3TWR) and ARC4:MCL1 (PDB ID: 3TWU) were obtained from Guettler et al. (Guettler, S. et al. Structural basis and sequence rules for substrate recognition by Tankyrase explain the basis for cherubism disease. Cell 147, 1340-1354, doi:10.1016/j.cell.2011.10.046 (2011)).

The ARC4 domain of human TNKS2 (PDB ID: 3TWU_A) and MCL1 peptide (PDB ID: 3TWU_B) were used as templates. The MCL1 peptide was substituted by the TBD1 (255-266 aa) or TBD2 (269-280 aa) peptide of human SOX9 and docked into a complex. The best predicted model for each of ARC4: SOX9 TBD1 and ARC4: SOX9 TBD2 was selected. The model structures were superimposed with ARC4:3BP2 and ARC4:MCL1 and visualized using the BIOVIA Discovery Studio Visualizer (http://accelrys.com/products/collaborative-science/biovia-discovery-studio/visualization.html).

Cycloheximide chase analysis. HEK293 cells were treated with 100 μg/ml of cycloheximide form Goldbio (C-930-1) for the indicated number of hours before lysis. Protein samples were subjected to SDS-PAGE to analyze protein stability.

Reporter gene assay. A firefly luciferase reporter plasmid with SOX9-dependent Col2a1 enhancer elements (Murakami, S., Lefebvre, V. & de Crombrugghe, B. Potent inhibition of the master chondrogenic factor Sox9 gene by interleukin-1 and tumor necrosis factor-alpha. J Biol Chem 275, 3687-3692 (2000)) was used to quantify the transcriptional activity of SOX9. To quantify β-catenin transcriptional activity, the TOPFlash reporter plasmid and recombinant mouse WNT-3a (PeproTech, 315-20) was used. Primary mouse articular chondrocytes or HEK293T cells were transfected with both a reporter plasmid and a constitutive Renilla luciferase plasmid. Cells were also treated with siRNAs or drugs as indicated. Renilla and firefly luciferase activity were sequentially measured using a Dual Luciferase Assay Kit (Promega). Renilla luciferase was used as a control.

List of SOX9 target genes in chondrocytes. Based on Oh et al. (SOX9 regulates multiple genes in chondrocytes, including genes encoding ECM proteins, ECM modification enzymes, receptors, and transporters. PLoS One 9, e107577, doi:10.1371/journal.pone.0107577 (2014)) genes with a log ₂(fold change) of <−2 after Sox9 deletion in mouse rib chondrocytes and associated with SOX9 ChIP-Seq peaks in mouse rib chondrocytes were selected as SOX9 target genes in chondrocytes. The SOX9 target genes in chondrocytes are listed in Table 7.

Generation of osteoarthritis-signature gene sets. Based on Dunn et al (Gene expression changes in damaged osteoarthritic cartilage identify a signature of non-chondrogenic and mechanical responses. Osteoarthritis Cartilage 24, 1431-1440, doi:10.1016/j.joca.2016.03.007 (2016)) genes with a |fold change| of >2 and a FDR q of <1×10⁻⁵ in damaged sites of articular cartilage compared to intact sites within the same patients with osteoarthritis were selected, and converted to mouse nomenclature using the biomaRt R package¹⁰². Genes Tables 9 and 10, respectively.

Preparation of hydrogels and in vivo confirmation of controlled release of embedded molecules. 6-O-Palmitoyl-l-ascorbic acid (76183) was purchased from Sigma-Aldrich. Hydrogels were prepared with 6-O-Palmitoyl-l-ascorbic acid as described previously (Zhang, S. et al. An inflammation-targeting hydrogel for local drug delivery in inflammatory bowel disease. Sci Transl Med 7, 300ra128, doi:10.1126/scitranslmed.aaa5657 (2015)). DiD percholate (5702) purchased from Tocris was loaded into the hydrogels and used for imaging of controlled release in mouse knee joints. PBS-suspended hydrogel (10 μl, PBS:hydrogel=1:1) containing 50 pmol DiD was administered intra-articularly, and at 1-9 days post-injection, light-emitting diode (LED) and fluorescence images of knee joints were obtained. LuminoGraph II (Atto) was used to acquire the images.

Experimental OA in mice. Eight-week-old male ICR mice were used for experimental OA. Experimental OA was induced by DMM (Destabilization of the medial meniscus) surgery on the right hindlimb, and sham surgery was conducted on the left hindlimb as a control (Glasson, S. S., Blanchet, T. J. & Morris, E. A. The surgical destabilization of the medial meniscus (DMM) model of osteoarthritis in the 129/SvEv mouse. Osteoarthritis Cartilage 15, 1061-1069, doi:10.1016/j.joca.2007.03.006 (2007)). 10 μl of PBS-suspended hydrogel (PBS:hydrogel=1:1) containing vehicle or 10 nmol drugs was administered intra-articularly.

Histology and immunohistochemistry. Mouse and rat knee joint samples and human cartilage samples were fixed with 4% paraformaldehyde overnight at 4° C. All samples were deprotected for 2-4 weeks 0.5M EDTA, pH 7.4 at 4° C. and embedded in paraffin. Mouse and rat paraffin blocks were sectioned at a thickness of 6 μm, and human paraffin blocks were sectioned to a thickness of 5 μm. For Safranin O staining, Alcian Blue/Fast Red staining, or immunostaining, sections were deparaffinized in xylene and hydrated using a graded ethanol series. All mouse histology images were acquired from medial tibial plateau except β-catenin immunostaining images where medial femoral condyle was used for imaging. To assess cartilage destruction in DMM mouse model, Safranin O stained samples were graded based on the Osteoarthritis Research Society International (OARSI) (Glasson, S. S., Chambers, M. G., Van Den Berg, W. B. & Little, C. B. The OARSI histopathology initiative—recommendations for histological assessments of osteoarthritis in the mouse. Osteoarthritis Cartilage 18 Suppl 3, S17-23, doi:10.1016/j.joca.2010.05.025 (2010)) by three blinded observers. On the basis of OARSI grading system, we primarily conducted integrative evaluation focusing on structural changes and proteoglycan loss in articular cartilage as a measure of cartilage destruction. OARSI grade 0-2 was classified as early stage and grade over 2 as OA late middle stage. Cartilage regeneration in osteochondral defect model was scored according to the International Cartilage Repair Society (ICRS) scoring system (Haikarainen, T., Krauss, S. & Lehtio, L. Tankyrases: structure, function and therapeutic implications in cancer. Curr Pharm Des 20, 6472-6488 (2014) and Mainil-Varlet, P. et al. Histological assessment of cartilage repair: a report by the Histology Endpoint Committee of the International Cartilage Repair Society (ICRS). J Bone Joint Surg Am 85-A Suppl 2, 45-57 (2003)) by three blinded observers.

Mouse limb-bud micromass culture. For the micromass culture of mesenchymal cells, limb-bud cells were isolated from E11.5 ICR mouse embryos. 2.0×10⁷ cells/ml were suspended in DMEM supplemented with 10% FBS, 100 units/ml penicillin, and 100 μg/ml streptomycin, and 15-μ1 drops were spotted on culture dishes. After 24 h, cells were treated as indicated for 3 days and subjected to Alcian Blue staining.

Chondrogenic differentiation of human mesenchymal stem cells. hMSCs were purchased from Lonza and Thermo Scientific. hMSCs were cultured in α-MEM supplemented with 20% FBS, 100 units/ml penicillin, 100 μg/ml streptomycin, and 250 ng/ml amphotericin B. To induce chondrogenesis, 2.5×10⁵ hMSCs were centrifuged to form a pellet in α-MEM supplemented with 20% FBS, 100 units/ml penicillin, 100 μg/ml streptomycin, and 250 ng/ml amphotericin B. After 3 days, the medium was changed to chondrogenic medium consisting of DMEM/F-12 supplemented with 100 units/ml penicillin, 100 μg/ml streptomycin, 250 ng/ml amphotericin B, 1.25 mg/ml BSA, 1% Insulin-Transferrin-Selenium, 1 mM Sodium pyruvate, 50 μM L-aspartic acid, 50 μM L-proline, 100 nM dexamethasone, and 10 ng/ml of TGF-β1 with or without indicated drugs. On day 21 (for drug treatment) or day 28 (for siRNA treatment), cells were harvested and subjected to Alcian Blue/Fast Red staining.

Generation of control shRNA-infected or shTNKS and shTNKS2-infected human mesenchymal stem cells. psPAX2 and pMD2.G were transfected to HEK293T. After 3 days, cell supernatants were harvested and filtered through a 0.45-μm filter. hMSCs were treated with 8 μg/ml polybrene and infected with the indicated lentiviruses. Twenty-four hours after infection, hMSCs were selected with 5 μg/ml puromycin and 200 μg/ml hygromycin for 4 days.

Rat osteochondral defect model. Twelve-week-old male Sprague Dawley rats were used as the osteochondral defect model. To expose the articular cartilage in the knee joints, a medial parapatellar incision was made and the patella was slightly displaced toward the medial condyle. A full-thickness cartilage defect (3 mm×1 mm×1 mm) was created using a 1-mm-diameter spherical drill at the surface of the femoral patellar groove. At the same time, hMSCs were suspended in 10 μl of fibrin glue (TISSEEL) by tapping, and implanted on the defect. To avoid immune rejection, cyclosporine A (C988900) from Toronto Research Chemicals was injected intra-peritoneally every day. At 8 weeks, rats were sacrificed for histological analyses.

Statistics. All experiments were carried out independently at least three times. All images are representative of at least three independent trials. For parametric tests, two-tailed Student's t test or one-way analysis of variance (ANOVA) followed by Fisher's least significant difference post-hoc test were used. For nonparametric tests, Mann-Whitney test or Kruskal-Wallis test followed by Mann-Whitney test were used. All statistical analysis was performed using IBM SPSS Statistics. A P-value <0.05 was considered statistically significant.

TABLE 1
List of siRNA

| | | | | SEQ |
|---|---|---|---|---|
| Gene | Strand | siRNA sequences | Species | ID NO |
| Tnks #1 | S | 5′-CACAGAGUCAC | Mouse | SEQ ID |
| | | ACUGACUAdTdT-3′ | | NO: 1 |
| | AS | 5′-UAGUCAGUGUG | | SEQ ID |
| | | ACUCUGUGdTdT-3′ | | NO: 2 |
| Tnks #2 | S | 5′-GUCUGUCGUUG | Mouse | SEQ ID |
| | | AGUACCUUdTdT-3′ | | NO: 3 |
| | AS | 5′-AAGGUACACAA | | SEQ ID |
| | | CGACAGACdTdT-3′ | | NO: 4 |
| Tnks #3 | S | 5′-ACAUAGCAGCG | Mouse | SEQ ID |
| | | UUACUGAUdTdT-3′ | | NO: 5 |
| | AS | 5′-AUCAGUAACGC | | SEQ ID |
| | | UGCUAUGUdTdT-3′ | | NO: 6 |
| Tnks2 #1 | S | 5′-CAGUGUAGUUU | Mouse | SEQ ID |
| | | UGAGUCUAdTdT-3′ | | NO: 7 |
| | AS | 5′-UAGACUCAAAA | | SEQ ID |
| | | CUACACUGdTdT-3′ | | NO: 8 |
| Tnks2 #2 | S | 5′-CUGUUCUGACU | Mouse | SEQ ID |
| | | GGUGACUAdTdT-3′ | | NO: 9 |
| | AS | 5′-UAGUCACCAGU | | SEQ ID |
| | | CAGAACAGdTdT-3′ | | NO: 10 |
| Tnks2 #3 | S | 5′-GUGUCUACUUG | Mouse | SEQ ID |
| | | UAUCACAUdTdT-3′ | | NO: 11 |
| | AS | 5′-AUGUGAUACAA | | SEQ ID |
| | | GUAGACACdTdT-3′ | | NO: 12 |
| Ctnnb1 #1 | S | 5′-GUUUUAGGCCU | Mouse | SEQ ID |
| | | GUUUGUAAdTdT-3′ | | NO: 13 |
| | AS | 5′-UUACAAACAGG | | SEQ ID |
| | | CCUAAAACdTdT-3′ | | NO: 14 |
| Ctnnb1 #2 | S | 5′-UCUGAACGUGC | Mouse | SEQ ID |
| | | AUUGUGAUdTdT-3′ | | NO: 15 |
| | AS | 5′-AUCACAAUGCA | | SEQ ID |
| | | CGUUCAGAdTdT-3′ | | NO: 16 |
| Ctnnb1 #3 | S | 5′-GUAAUCUGGAG | Mouse | SEQ ID |
| | | ACGUGUAAdTdT-3′ | | NO: 17 |
| | AS | 5′-UUACACGUCUC | | SEQ ID |
| | | CAGAUUACdTdT-3′ | | NO: 18 |
| Rnf146 #1 | S | 5′-CAGAUACCUCC | Mouse | SEQ ID |
| | | GUUGAAGAdTdT-3′ | | NO: 19 |
| | AS | 5′-UCUUCAACGGA | | SEQ ID |
| | | GGUAUCUGdTdT-3′ | | |
| | | | | NO: 20 |
| Rnf146 #2 | S | 5′-CUCUAGAGCAU | Mouse | SEQ ID |
| | | CACAGCUUdTdT-3′ | | NO: 21 |
| | AS | 5′-AAGCUGUGAUG | | SEQ ID |
| | | CUCUAGACrdTdT-3′ | | NO: 22 |
| Rnf146 #3 | S | 5′-GUCGACAAGAG | Mouse | SEQ ID |
| | | AUUCCUGAdTdT-3′ | | NO: 23 |
| | AS | 5′-UCAGGAAUCUC | | SEQ ID |
| | | UUGUCGACdTdT-3′ | | NO: 24 |
| TNKS | S | 5′-GCAUGGAGCUU | Human | SEQ ID |
| | | GUGUUAAUUU-3′ | | NO: 25 |
| | AS | 5′-AUUAACACAAG | | SEQ ID |
| | | CUCCAUGCUU-3′ | | NO: 26 |
| TNKS2 | S | 5′-GGAAAGACGUA | Human | SEQ ID |
| | | GUUGAAUAUU-3′ | | NO: 27 |
| | AS | 5′-UAUUCAACUAC | | SEQ ID |
| | | GUCUUUCCUU-3′ | | NO: 28 |
| Sox9 #1 | S | 5′-GUAAAGGAAGG | Mouse | SEQ ID |
| | | UAACGAUUdTdT-3′ | | NO: 29 |
| | AS | 5′-AAUCGUUACCU | | SEQ ID |
| | | UCCUUUACdTdT-3′ | | NO: 30 |
| Sox9 #2 | S | 5′-GAGACAUCGGA | Mouse | SEQ ID |
| | | CAGACCUUdTdT-3′ | | NO: 31 |
| | AS | 5′-AAGGUCUGUCC | | SEQ ID |
| | | GAUGUCUCdTdT-3′ | | NO: 32 |
| Sox9 #3 | S | 5′-GUUUGUUUCCC | Mouse | SEQ ID |
| | | UCUCCAAAdTdT-3′ | | NO: 33 |
| | AS | 5′-UUUGGAGAGGG | | SEQ ID |
| | | AAACAAACdTdT-3′ | | NO: 34 |

TABLE 2
List of PCR Primers

| | | Primer | | SEQ ID |
|---|---|---|---|---|
| Gene | Strand | sequences | Species | NO |
| Hprt | S | 5′-AGTCCCAGCG | Mouse | SEQ ID |
| | | TCGTGATTAG-3′ | | NO: 35 |
| | AS | 5′-GTATCCAACAC | | SEQ ID |
| | | TTCGAGAGGTC-3′ | | NO: 36 |
| Tnks1 | S | 5′-GAAGGAAGGA | Mouse | SEQ ID |
| | | GAAGTTGCGG-3′ | | NO: 37 |
| | AS | 5-AATGAAAGGAG | | SEQ ID |
| | | AACCGTGGAAC-3′ | | NO: 38 |
| Tnks2 | S | 5′-CGGCGTCTTC | Mouse | SEQ ID |
| | | AACAGATACA-3′ | | NO: 39 |
| | AS | 5′-AGCCATCAAC | | SEQ ID |
| | | CATACCTTCAG-3′ | | NO: 40 |
| Col2a1 | S | 5′-ACCTTGGACG | Mouse | SEQ ID |
| | | CCATGAAAGT-3′ | | NO: 41 |
| | AS | 5′-CGGGAGGTCT | | SEQ ID |
| | | TCTGTGATCG-3′ | | NO: 42 |
| Comp | S | 5′-GTAAACACCG | Mouse | SEQ ID |
| | | CCACTGATGA-3′ | | NO: 43 |
| | AS | 5′-TGGGAGAAGC | | SEQ ID |
| | | AGAAGACACC-3′ | | NO: 44 |
| Col9a2 | S | 5-GATGGGTCCTC | Mouse | SEQ ID |
| | | GTGGCTAT-3′ | | NO: 45 |
| | AS | 5′-GTTCCCTTTG | | SEQ ID |
| | | GGCCTGTTAT-3′ | | NO: 46 |
| Col6a3 | S | 5′-TTATGGTGCT | Mouse | SEQ ID |
| | | GATGTTGACTGG-3′ | | NO: 47 |
| | AS | 5′-ATTGCTGTTG | | SEQ ID |
| | | GTTTGGTCGTT-3′ | | NO: 48 |
| Acan | S | 5′-CCCAAGCACA | Mouse | SEQ ID |
| | | GAGGTAAACAG-3′ | | NO: 49 |
| | AS | 5′-CTCACATTGC | | SEQ ID |
| | | TCCTGGTCTG-3′ | | NO: 50 |
| Dcn | S | 5′-AGGCTTCCTA | Mouse | SEQ ID |
| | | CTCGGCTGTGA-3′ | | NO: 51 |
| | AS | 5′-GTTCGGCGGC | | SEQ ID |
| | | ATTTGACTTT-3′ | | NO: 52 |
| Col6a1 | S | 5′-TGAAAATGTG | Mouse | SEQ ID |
| | AS | CTCCTGCTGTG-3′ | | NO: 53 |
| | | 5′-TGTCCCGTTG | | SEQ ID |
| | | AGTGTCAGAA-3′ | | NO: 54 |
| Col9a1 | S | 5′-AGCTGATGGA | Mouse | SEQ ID |
| | | TTAACAGGACC-3′ | | NO: 55 |
| | AS | 5′-TTCCCAGGGT | | SEQ ID |
| | | CTCCAATAGG-3′ | | NO: 56 |
| Bgn | S | 5′-GCATTGAGAT | Mouse | SEQ ID |
| | | GGGCGGGAA-3′ | | NO: 57 |
| | AS | 5′-AGTAGGGCAC | | SEQ ID |
| | | AGGGTTGTTG-3′ | | NO: 58 |
| Chad | S | 5′-ACAACCGCCT | Mouse | SEQ ID |
| | | GAACCAACT-3′ | | NO: 59 |
| | AS | 5-GGGGAGGGATT | | SEQ ID |
| | | CTGTGTCTT-3′ | | NO: 60 |
| Matn3 | S | 5′-CAGTGTGAGG | Mouse | SEQ ID |
| | | GGTTTCTG-3′ | | NO: 61 |
| | AS | 5′-AGCACCATAA | | SEQ ID |
| | | GTTCATAGCC-3′ | | NO: 62 |
| Ctnnb1 | S | 5′-CCACAGGATT | Mouse | SEQ ID |
| | | ACAAGAAGCGG-3′ | | NO: 63 |
| | AS | 5′-CCATTCCCAC | | SEQ ID |
| | | CCTACCAAGT-3′ | | NO: 64 |
| Rnf146 | S | 5′-AGCACAGAGA | Mouse | SEQ ID |
| | | ATGAACCAGCA-3′ | | NO: 65 |
| | AS | 5′-TGAAGCACCC | | SEQ ID |
| | | TTTACACACAGA-3′ | | NO: 66 |
| Sox9 | S | 5′-AAGATGACCG | Mouse | SEQ ID |
| | | ACGAGCAGGA-3′ | | NO: 67 |
| | AS | 5′-ATGTGAGTCT | | SEQ ID |
| | | GTTCCGTGGC-3′ | | NO: 68 |
| HPRT1 | S | 5′-CCTGGCGTCG | Human | SEQ ID |
| | | TGATTAGTG-3′ | | NO: 69 |
| | AS | 5′-CTTGCGACCT | | SEQ ID |
| | | TGACCATCTTT-3′ | | NO: 70 |
| TNKS1 | S | 5′-TCAGGGAACG | Human | SEQ ID |
| | | ATTTTGCTGGA-3′ | | NO: 71 |
| | AS | 5′-ACTCTGGGTA | | SEQ ID |
| | | TGCCTGTTCTC-3′ | | NO: 72 |
| TNKS2 | S | 5′-GCGATACCCA | Human | SEQ ID |
| | AS | AGGCAGACATT-3′ | | NO: 73 |
| | | 5′-AACAAGAGGG | | SEQ ID |
| | | CAGAGCAGATGG-3′ | | NO: 74 |

TABLE 3
List of PCR primers used for subcloning

| | | | | | | SEQ |
|---|---|---|---|---|---|---|
| | | Primer | Enzyme | | | ID |
| Gene | Strand | sequences | Sites | Species | Plasmid | NO |
| SOX9 | S | 5′-CCGAATTCA | EcoRI | Human | pcDNA3- | SEQ |
| | | TGAATCTCCTGG | XbaI | | HA- | ID |
| | | ACCCCTTC-3′ | | | SOX9 | NO: |
| | | | | | | 75 |
| | AS | 5′-CGTCTAGAT | | | | SEQ |
| | | CAAGGTCGAGTG | | | | ID |
| | | AGCTGTGT-3′ | | | | NO: |
| | | | | | | 76 |
| SOX9 | S | 5′-AAGAATTCG | EcoRI | Human | Pcmv10- | SEQ |
| | | AATCTCCTGGAC | XbaI | | 3xFLAG- | ID |
| | | CCCTTCAT-3′ | | | | NO: |
| | | | | | | 77 |
| | AS | 5′-CGTCTAGAT | | | SOX9 | SEQ |
| | | CAAGGTCGAGTG | | | | ID |
| | | AGCTGTGT-3′ | | | | NO: |
| | | | | | | 78 |
| SOX9 | S | 5′-AAGCTAGCA | NileI | Human | pTK- | SEQ |
| | | ACCATGGACTAC | XbaI | | 3xFLAG- | ID |
| | | AAAGACCA-3′ | | | | NO: |
| | | | | | | 79 |
| | AS | 5-CGTCTAGATC | | | SOX9 | SEQ |
| | | AAGGTCGAGTGA | | | | ID |
| | | GCTGTGT-3′ | | | | NO: |
| | | | | | | 80 |
| TNKS2 | S | 5′-AAAAGCTTG | HindIII | Human | pEGFP- | SEQ |
| | | GATCATGTCGGG | BamHI | | TNKS2 | ID |
| | | TCGCCGCTG-3′ | | | | NO: |
| | | | | | | 81 |
| | AS | 5′-AAGGATCCT | | | | SEQ |
| | | TATCCATCGACC | | | | ID |
| | | ATACCTTCAGG | | | | NO: |
| | | CCTCATAA-3′ | | | | 82 |

TABLE 4
List of PCR primers used for mutagenesis

| | | | Muta- | | |
|---|---|---|---|---|---|
| | | Primer | genesis | Spe- | SEQ ID |
| Gene | Strand | sequences | Site | cies | NO |
| SOX9 | S | 5′-CAGCCCCCTATC | ΔTBD1 | Human | SEQ ID |
| | | GACTTCCGCGA-3′ | 772- | | NO: 83 |
| | AS | 5′ CCCCTCTCGCT | 792 | | SEQ ID |
| | | TCAGGTCAGCCT-3′ | bp | | NO: 84 |
| SOX9 | S | 5′-AGCAGCGACGT | ΔTBD2 | Human | SEQ ID |
| | | CATCTCCAACAT-3′ | 814- | | NO: 85 |
| | AS | 5′-GAAGTCGATAG | 834 | | SEQ ID |
| | | GGGGCTGTCT-3′ | bp | | NO: 86 |
| SOX9 | S | 5′-AGCAGCGACGT | ΔTBD1/2 | Human | SEQ ID |
| | | CATCTCCAACAT-3′ | 772- | | NO: 87 |
| | AS | 5′-CCCCTCTCGCT | 834 | | SEQ ID |
| | | TCAGGTCAGCCT-3′ | bp | | NO: 88 |
| SOX9 | S | 5′ CCCTTGCCAGA | R257A | Human | SEQ ID |
| | | GGGGGGCA-3′ | | | NO: 89 |
| | AS | 5′-TGCCCCCTCTCG | | | SEQ ID |
| | | CTTCAGGTCA-3′ | | | NO: 90 |
| SOX9 | S | 5′-GACGTGGACATC | R271A | Human | SEQ ID |
| | | GGCGAGCTGA-3′ | | | NO: 91 |
| | AS | 5′-TGCGAAGTCGAT | | | SEQ ID |
| | | AGGGGGCTGTCT-3′ | | | NO: 92 |

TABLE 5
List of PCR primers used for
shRNA plasmid construction

| | | | | SEQ ID |
|---|---|---|---|---|
| Gene | Strand | Primer sequences | Species | NO |
| Control | s | 5′-CCGGAAACAAGATGAAG | | SEQ ID |
| | | AGCACCAACTCGAGTTGGT | | NO: 93 |
| | | GCTCTTCATCTTGTTTTTT | | |
| | | TTG-3′ | | |
| | AS | 5′-AATTCAAAAAAAACAAG | | |
| | | ATGAAGAGCACCAACTCGAG | | SEQ ID |
| | | TTGGTGCTCTTCATCTTGTT | | NO: 94 |
| | | T-3′ | | |
| Tnks | S | 5′-CCGGGCTAGATGTGTTG | Mouse | SEQ ID |
| | | GCTGATATCTCGAGATATC | | NO: 95 |
| | | AGCCAACACATCTAGCTT | | |
| | | TTTG-3′ | | |
| | AS | 5′-AATTCAAAAAGCTAGA | | |
| | | TGTGTTGGCTGATATCTCG | | SEQ ID |
| | | AGATATCAGCCAACACATC | | NO: 96 |
| | | TAGC-3′ | | |
| Tnks2 | S | 5′-CCGGCATCGACACAAGC | | SEQ ID |
| | | TGATTAAACTCGAGTTTAAT | | NO: 97 |
| | | CAGCTTGTGTCGATGTTTTT | | |
| | | G-3′ | | |
| | AS | 5′-AATTCAAAAACATCGAC | Mouse | |
| | | ACAAGCTGATTAAACTCGA | | SEQ ID |
| | | GTTTAATCAGCTTGTGTC | | NO: 98 |
| | | GATG-3′ | | |
| Rnf146 | S | 5′-CCGGATTTCTGCCCAC | Mouse | SEQ ID |
| | | GTAACATTACTCGAGTAAT | | NO: 99 |
| | | GTTACGTGGGCAGAAATTT | | |
| | | TTTG-3′ | | |
| | AS | 5′-AATTCAAAAAATTTCTG | | |
| | | CCCACGTAACATTACTCGA | | SEQ ID |
| | | GTAATGTTACGTGGGCAG | | NO: 100 |
| | | AAAT-3′ | | |
| TNKS | S | 5′-CCGGGCCCATAATGAT | Human | SEQ ID |
| | | GTCATGGAACTCGAGTTCC | | NO: 101 |
| | | ATGACATCATTATGGGCTT | | |
| | | TTTG-3′ | | |
| | AS | 5′-AATTCAAAAAGCCCAT | | SEQ ID |
| | | AATGATGTCATGGAACTC | | NO: 102 |
| | | GAGTTCCATGACATCATT | | |
| | | ATGGGC-3′ | | |
| TNKS2 | S | 5′-CCGGAAGGAAAGACGT | Human | SEQ ID |
| | | AGTTGAATACTCGAGTATT | | NO: 103 |
| | | CAACTACGTCTTTCCTTTT | | |
| | | TTTG-3′ | | |
| | AS | 5′-AATTCAAAAAAAGGAA | | SEQ ID |
| | | AGACGTAGTTGAATACTCG | | NO: 104 |
| | | GATATTCAACTACGTCTTT | | |
| | | CCTT-3′ | | |

TABLE 6
List of mouse proteins involved in IPA chondrogenesis
Proteins involved in chondrogenesis (52 proteins)

| | | | | | |
|---|---|---|---|---|---|
| ALG2 | CR3L2 | GRN | NFKB2 | Q9DAB5 | SOX12 |
| BMAL1 | CREB1 | GSK3A | NKX32 | REL | SOX4 |
| BMP2 | CTNB1 | GSK3B | PDGFA | RELB | SOX9 |
| BMP4 | CYR61 | HHAT | PER1 | RHOA | TF65 |
| BMR1B | DHH | HIF1A | PP2BA | SHH | TNF12 |
| CANB1 | ENPP1 | HMGB2 | PP2BB | SIR1 | VNN1 |
| CANB2 | FGF18 | IHH | PP2BC | SMAD3 | WNT3A |
| CBP | FGFR3 | NFAC3 | PRGC1 | SOMA | |
| CHP1 | GDF5 | NFKB1 | PTHR | SOX11 | |

TABLE 7
List of target gene of SOX9 in chondrocytes
SOX9 target genes in chondrocytes (91 genes)

| | | | | | |
|---|---|---|---|---|---|
| Acan | Col9a2 | Fzd9 | Mgp | Rab11fip4 | Susd5 |
| Aldh1l2 | Col9a3 | Gfpt1 | Mia | Rhbdd1 | Tprgl |
| Alx1 | Colgalt2 | Gls | Mtss1l | Rnf144a | Trib3 |
| Arsi | Cox17 | Got1 | Ncmap | Rtkn | Trim47 |
| Atf4 | Cp | Grb2 | Ndufa2 | Scin | Trpv4 |
| B230206H07Rik | Cpm | Hip1r | Oat | Sdk2 | Ucma |
| B4galnt3 | D630045J12Rik | Hr | Papss2 | Slc1a5 | WSCD2 |
| Bcat1 | Dnttip1 | Kcns1 | Pck2 | Slc26a2 | Wwp2 |
| Bmp6 | Enpp2 | Lcn2 | Pcolce2 | Slc38a3 | Xylt1 |
| Chadl | Extl1 | Ldlrad3 | Pde4dip | Slc39a14 | Zfp385b |
| Chst11 | Fam89a | Lect1 | Phyh | Smpd3 | Zfp385c |
| Cmklr1 | Fbxo7 | Leprel1 | Plxnb1 | Snorc | |
| Col11a1 | Fgfr3 | Lgals3 | Ppp1r1b | Sobp | |
| Col27a1 | Fgfrl1 | Loxl4 | Ppp2ca | Sox6 | |
| Col2a1 | Foxd1 | Matn3 | Prdx5 | Spats2l | |
| Col9a1 | Fry | Mgat4a | Prelp | Stk39 | |

TABLE 8
Cartilage-signature genes
Cartilage-signature genes (235 genes)

| | | | | | | | | |
|---|---|---|---|---|---|---|---|---|
| 3632451O06Rik | Cd14 | Dio2 | Fzd9 | Lect1 | Nptx1 | Scrg1 | Sort1 | Zdbf2 |
| 4930523C07Rik | Cdkn1a | Dnajb9 | Gab1 | Lipo3 | Nr4a2 | Scube3 | Sox5 | Zfp385b |
| A2m | Cgref1 | Ecm2 | Gfpt2 | Loxl4 | Nr4a3 | Sdk2 | Sox6 | Zim1 |
| Acan | Chac1 | Edil3 | Gjc3 | Matn1 | Nt5e | Sec16b | Sox9 | |
| Adamts3 | Chad | Efcab1 | Glis3 | Matn3 | Omd | Sema3e | Sparcl1 | |
| Adcy2 | Chadl | Egr1 | Gm22 | Mdfi | Panx3 | Sema6a | Srgap1 | |
| Adgrg1 | Chrdl1 | Egr2 | Gm39701 | Mertk | Papss2 | Serinc5 | Srgn | |
| Airn | Chst11 | Ehd3 | Gm7265 | Mfi2 | Pcsk6 | Sim2 | Srxn1 | |
| Ak4 | Clec3a | Eng | Gprc5a | Mfsd7c | Pde3a | Slc16a2 | Stk26 | |
| Alx1 | Cmklr1 | Enpp1 | Gpx3 | Mgat4a | Perp | Slc16a4 | Stk32b | |
| Angptl1 | Cmtm5 | Enpp2 | Grb10 | Mia | Phxr4 | Slc1a1 | Stk40 | |
| Arc | Col10a1 | Epas1 | Gstk1 | Mir377 | Pla2g5 | Slc1a5 | Sulf2 | |
| Arl5b | Col11a1 | Epyc | Hapln1 | Mir411 | Plcd1 | Slc22a23 | Tcn2 | |
| Asb4 | Col11a2 | Ern1 | Hist1h1c | Mir505 | Plet1 | Slc22a4 | Tet1 | |
| Atf3 | Col2a1 | Extl1 | Hivep2 | Mir568 | Plod2 | Slc25a36 | Tet2 | |
| Atp1b2 | Col9a1 | F13a1 | Hpgd | Moxd1 | Prg4 | Slc26a2 | Tgfb2 | |
| Auts2 | Col9a2 | Fabp7 | Igsf9b | Mpzl2 | Prkg2 | Slc2a10 | Tmbim1 | |
| B4galnt3 | Col9a3 | Fam180a | Il16 | Mt2 | Prss35 | Slc38a3 | Tmem56 | |
| Baiap2l1 | Colgalt2 | Fam19a5 | Islr | Mtap7d3 | Ptger1 | Slc6a12 | Tnfrsf21 | |
| BC026585 | Comp | Fam46a | Itga10 | Mtss1l | Rab11fip4 | Slc7a11 | Tns2 | |
| Bdh1 | Cpm | Fbln7 | Kank1 | Mustn1 | Rbp4 | Slc7a3 | Tram2 | |
| Bmp2 | Cpxm2 | Fgfr2 | Kcna6 | Ncmap | Rcan1 | Slc8a3 | Trp53inp2 | |
| Bmp5 | Creb3l2 | Fgfr3 | Kcnk1 | Ndrg2 | Rgs2 | Smox | Trps1 | |
| Bmp6 | Crispld1 | Fmod | Kcnma1 | Nebl | Rin2 | Smpdl3a | Trpv4 | |
| Btg2 | Cspg4 | Fos | Kdm6b | Nfatc1 | Rnf144b | Snora23 | Ucma | |
| C1qtnf3 | Cthrc1 | Fosb | Kdm7a | Nfatc2 | S100a1 | Snora28 | Wisp3 | |
| C4b | Ctsh | Frmd4b | Kif21a | Ngf | S100b | Snorc | Xist | |
| Car6 | Cybrd1 | Fry | Klhl13 | Ninj1 | Scara3 | Snord82 | Xylt1 | |
| Cd109 | Cytl1 | Frzb | Klk10 | Ninj2 | Scin | Sobp | Zbtb20 | |

TABLE 9
Unregulated genes in osteoarthritic cartilage
Upregulated genes in osteoarthritic cartilage (150 genes)

| | | | | | |
|---|---|---|---|---|---|
| 3830406C13Rik | Cenpk | Fam167a | Kcnn4 | Pcdh10 | St6galnac5 |
| Abracl | Cep55 | Fam60a | Kcns3 | Pcdh18 | Stx1a |
| Adamts14 | Chst13 | Fat3 | Kif20a | Pgm2l1 | Syt11 |
| Adamts5 | Cited4 | Fgf9 | Lamb3 | Plaur | Sytl2 |
| Adamts6 | Ckb | Fhl2 | Lif | Plekhg1 | Tbx3 |
| Adgrg1 | Clic3 | Foxf1 | Lmo2 | Popdc3 | Tenm3 |
| Adtrp | Col13a1 | Fstl3 | Lrrc8c | Postn | Tfpi |
| AI661453 | Col18a1 | Fzd10 | Lrrc8e | Prex2 | Tgfbi |
| Akr1c20 | Col1a1 | Galnt7 | Lum | Ptges | Tmem100 |
| Anln | Col7a1 | Gja1 | Map1b | R3hdml | Tmem119 |
| Arhgap44 | Cpeb2 | Gjb2 | Mob3b | Rab23 | Tmem200a |
| Arl4a | Csdc2 | Glis3 | Moxd1 | Rcan1 | Tmem59l |
| Arntl2 | D330045A20Rik | Glrb | Msx2 | Rhbdl2 | Tnfaip6 |
| Aspm | Diras1 | Gmnn | Mtss1 | S100a4 | Tnfrsf12a |
| Aspn | Dkk3 | Gpc4 | Ncapg | Sema3c | Tom1l1 |
| Atrnl1 | Dnajc12 | Gria2 | Nedd4l | Serpine1 | Top2a |
| B3gnt2 | Dner | Hey2 | Nedd9 | Serpine2 | Trim36 |
| B3gnt5 | Dsg2 | Hhipl1 | Ngf | Sgk1 | Uroc1 |
| Bmpr1b | Dusp4 | Hmga2 | Nt5e | Sik1 | Vcan |
| C1galt1 | Ebf3 | Homer2 | Ntf3 | Slc2a5 | Veph1 |
| Car12 | Egr2 | Hunk | Ociad2 | Slc38a5 | Vwc2 |
| Cdk1 | Epha3 | Ier3 | Ogn | Slc6a6 | Wisp1 |
| Cdkn2b | Eva1a | Iqgap3 | Osbpl3 | Slitrk6 | Wnt5a |
| Cdkn3 | Evi2a | Itga3 | P3h2 | Sntb1 | Zfp365 |
| Cenpf | Fam132b | Kcne4 | Pamr1 | Sqrdl | Zfp367 |

TABLE 10
Downregulated genes in osteoarthritic cartilage
Downregulated genes in osteoarthritic cartilage (71 genes)

| | | | | | |
|---|---|---|---|---|---|
| Agtr2 | Cmya5 | Fbln7 | Lgi4 | Ptger3 | Srl |
| A1x4 | Col11a2 | Fgf14 | Lrrtm2 | Rarres2 | Steap4 |
| Apol9b | Col16a1 | Frzb | Mpped2 | Rcan2 | Stk32b |
| Atp1b2 | Crim1 | Gpc5 | Myh14 | Rflna | Tac1 |
| C530008M17Rik | Cyp39a1 | Gprc5b | Myoz3 | Rspo3 | Tceal5 |
| Cacna1c | Dact1 | Grin2c | Nfam1 | Sdc3 | Tmem176a |
| Cacna2d2 | Dcc | Gucy1a3 | Nrxn2 | Sez6l | Tmem176b |
| Capn6 | Ddit4 | Hmgcll1 | Obscn | Sgsm1 | Tnfrsf4 |
| Cdhr1 | Erich3 | Igf2 | Pde3b | Slc14al | Wnk2 |
| Ces1a | Esr1 | Il17rb | Piezo2 | Slc25a27 | Zcchc5 |
| Chrdl2 | Evx1 | Il18bp | Ppp1r1b | Slitrk4 | Zfp385c |
| Cmtm5 | Fam198a | Kif1a | Prx | Sncg | |

### Example 1. Identification of a Regulatory Factor that Governs Cartilage Matrix Anabolism

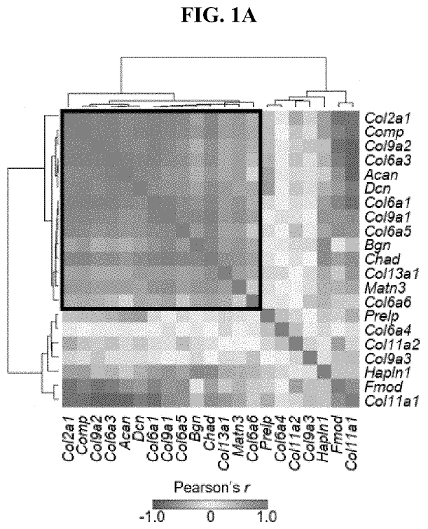

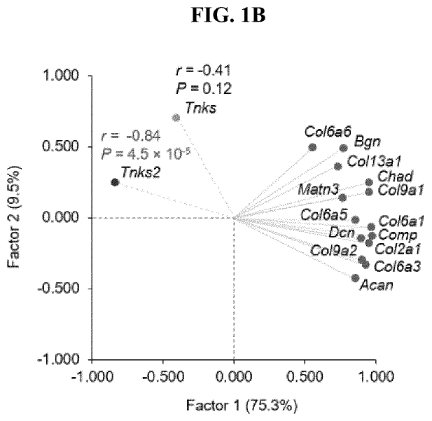

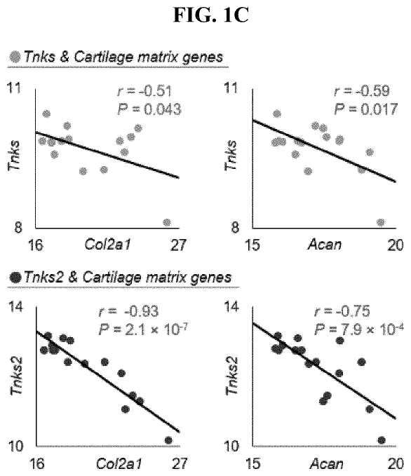

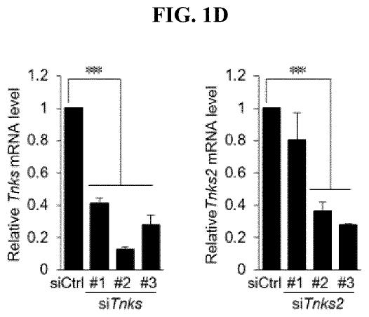

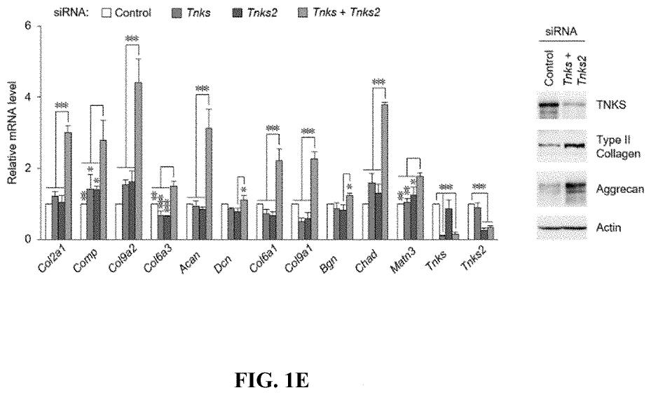

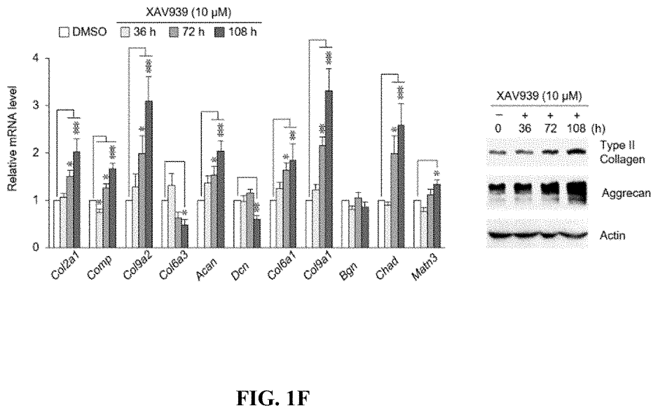

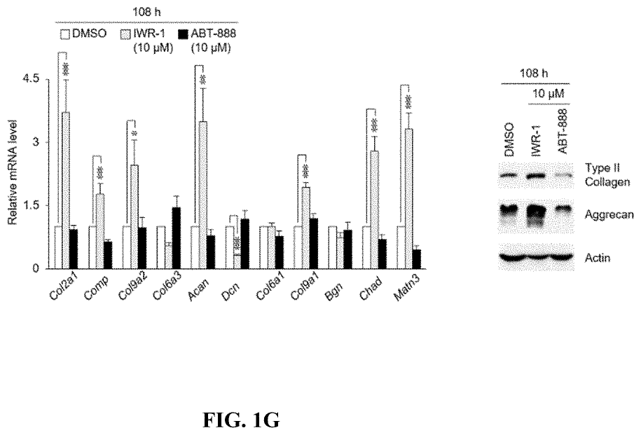

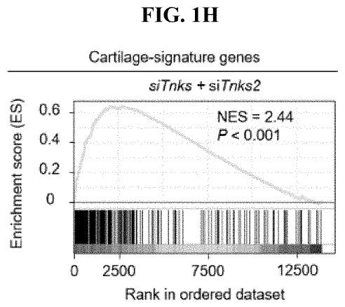

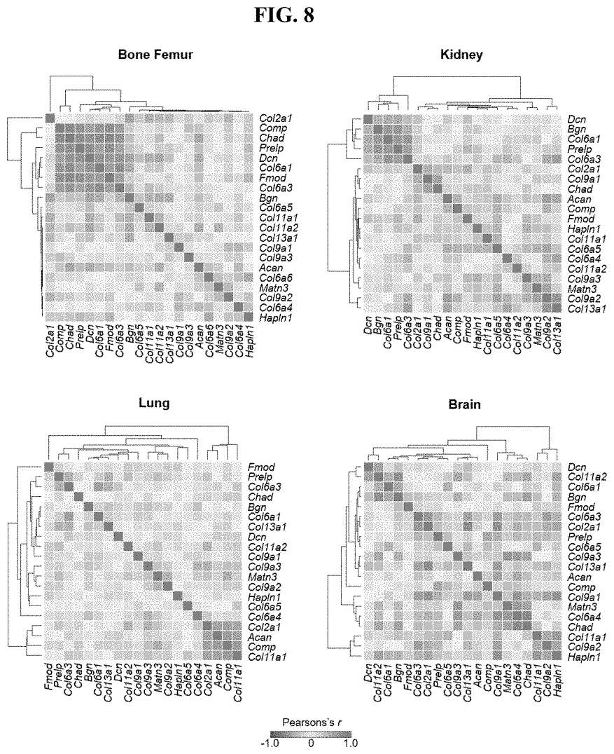

To screen for a key regulatory factor that could be targeted to stimulate cartilage matrix anabolism, genetic analysis on transcriptomes of mouse reference populations using post-hoc factor analysis were conducted. First, we assessed the transcriptional variance in the cartilage tissues of 16 strains of BXD mice. We noted that, among 21 cartilage matrix genes listed up by Heinegard and Saxne (The role of the cartilage matrix in osteoarthritis. Nat Rev Rheumatol 7, 50-56, doi:10.1038/nrrheum.2010.198 (2011)), 14 cartilage matrix genes showed strong positive correlation in their transcript abundance (FIG. 1a). These high correlations were absent in organs without cartilaginous functions, such as bone femur, kidney, lung, and brain (FIG. 8). We then attempted to extract a common axis underlying cartilage anabolism by performing a principal component analysis on 14 highly inter-correlated cartilage matrix genes (see black box in FIG. 1a). The first axis identified (Factor 1) essentially reflects the state of cartilage matrix anabolism (FIG. 1b). We then computed Pearson's correlation coefficients between these 14 cartilage matrix genes and Factor 1 genes (Factor I and transcription factors, enzymes and various gene identified as signal molecules with unknown functions in cartilage). Tankyrase showed striking negative correlations with the anabolic axis and with individual cartilage matrix genes and was therefore, investigated further (FIG. 1b, c). Tankyrase showed striking negative correlations with the anabolic axis and with individual cartilage matrix genes and was therefore selected as a candidate and investigated further (FIG. 1b, c).

We then examined the potential regulatory role of tankyrase in cartilage anabolism. Knockdown of both Tnks and Tnks2 collectively induced the expression of cartilage-specific matrix genes in primary cultured mouse chondrocytes (FIG. 1d, e). On the other hand, the individual knockdown of Tnks or Tnks2 failed to increase the cartilage matrix anabolism, suggestive of the redundant roles of tankyrase-1 and -2 in this regulation (FIG. 1e). Treatment with XAV939 or IWR-1, highly specific and potent TNKS/2 inhibitors also increased the expression of cartilage-specific matrix genes in chondrocytes (FIG. 1f, g). However, the PARP1/2 inhibitor, ABT-888, failed to increase their expressions. PARP is a member of the family with PARylation activity. PARP 1 to PARP 16 are known and TNK1 and TNK2 is PARP-5a, and PARP-5b, respectively. Thus the above result indicates clearly that only TNKS inhibition among PARP can induce the cartilage matrix specific gene expression since XAV939 is a TNKS inhibitor and ABT-888 is a PARP1/2 inhibitor. ABT-888 used as a negative control.

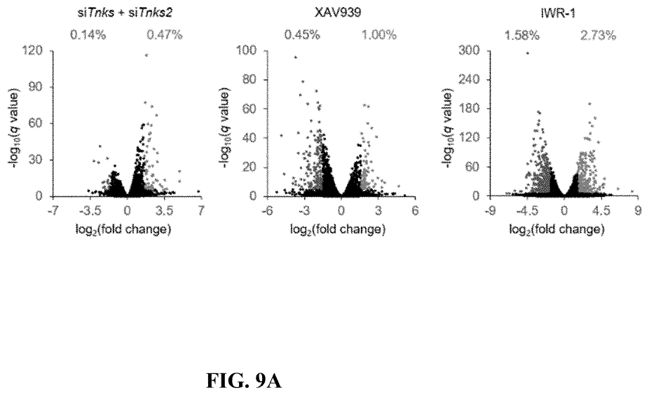

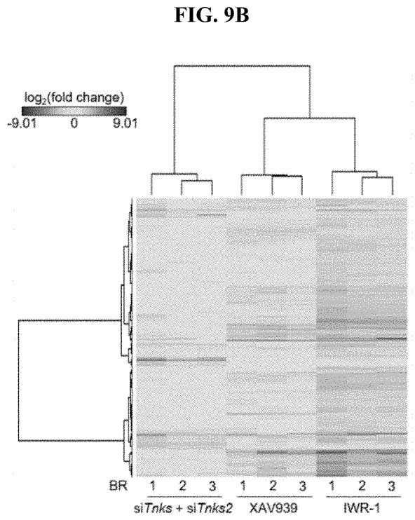

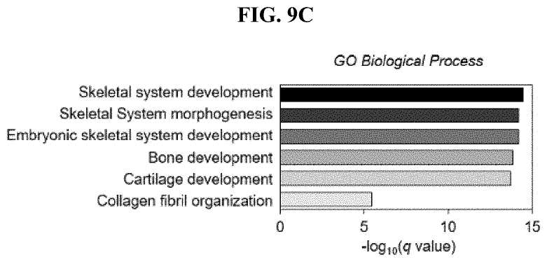

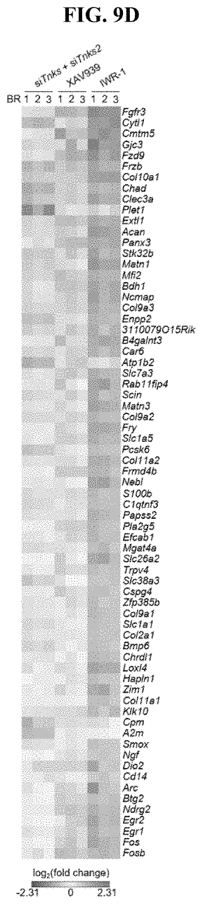

To comprehensively elucidate the effect of tankyrase inhibition at the whole transcriptome level, we performed RNA sequencing for chondrocytes treated with siRNAs targeting Tnks and Tnks2, XAV939, or IWR-1(FIG. 9a). As a result, all three tankyrase inhibition group compared to their respective control groups showed similar group of differentially expressed genes up or downregulated (FIG. 9b). GO analysis of the commonly upregulated genes in response to tankyrase inhibition revealed a strong association with terms related to cartilage development (FIG. 9c). Next, we generated a comprehensive list of cartilage-signature genes by utilizing public transcriptome datasets. Tankyrase inhibition induced strong transcription of key cartilage-identity genes (FIG. 9d). In addition, gene set enrichment analysis (GSEA) revealed that cartilage-signature genes were positively enriched in the whole transcriptome obtained from chondrocytes treated with siTnks and siTnks2 or tankyrase inhibitors, XAV939 and IWR-1(FIG. 1h, j). Thus, tankyrase inhibition promotes cartilage matrix anabolism and strengthens overall chondrogenic features in chondrocytes

### Example 2. Identification that SOX9 Interacts with Tankyrase Through its Conserved Tankyrase-Binding Domains

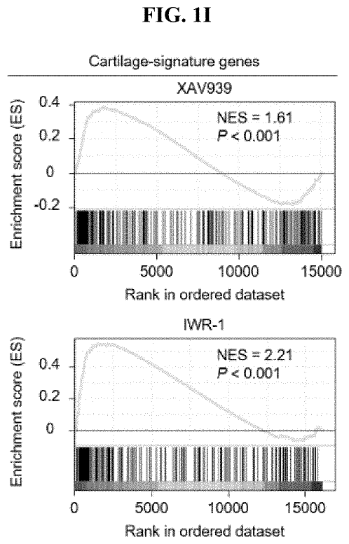

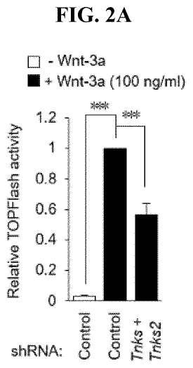

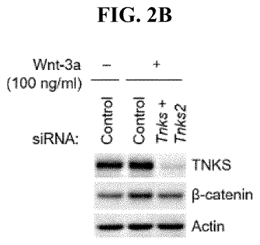

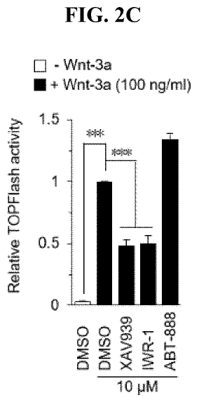

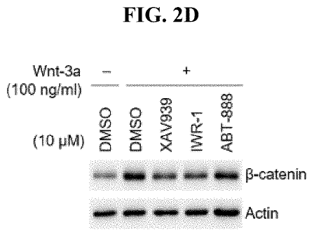

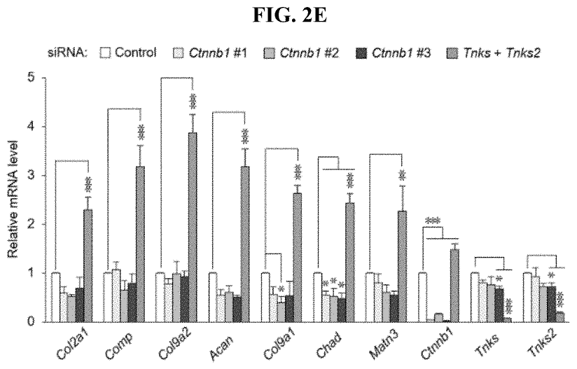

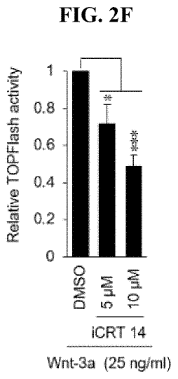

Here it was discovered that SOX9 interacts with tankyrase through its conserved tankyrase-binding domains. To understand the molecular mechanism underlying the effect of tankyrase inhibition on cartilage anabolism, we aimed to identify tankyrase substrates responsible for the regulation of cartilage matrix genes. Axin, a well-established target of tankyrase, is subjected to proteasomal degradation upon PARylation-dependent ubiquitination (Huang, S. M. et al. Tankyrase inhibition stabilizes axin and antagonizes Wnt signalling. Nature 461, 614-620, doi:10.1038/nature08356 (2009), Zhang, Y. et al. RNF146 is a poly(ADP-ribose)-directed E3 ligase that regulates axin degradation and Wnt signalling. Nat Cell Biol 13, 623-629, doi:10.1038/ncb2222 (2011)). Consistently, tankyrase inhibition reduced β-catenin stability and activity in chondrocytes (FIG. 2a-d). However, when transcription inhibitor iCRT 1429 responsive to β-catenin or Ctnnb1 siRNA, it was found that they did not significantly affect the expression of cartilage matrisome. This indicates that β-catenin is not involved in the tankyrase inhibition in cartilage anabolism (FIG. 2e, f, g).

To find a novel tankyrase-binding substrate that regulates cartilage matrix anabolism, we performed liquid chromatography-tandem mass spectrometry (LC-MS/MS) analysis for the proteome co-immunoprecipitated with the endogenous tankyrase in chondrocytes (FIG. 3a and Table 10). We considered proteins that are detected in more than one biological replicate as putative tankyrase-interacting proteins (FIG. 3b). Among these binding partners, candidate substrates were further screened using a tankyrase-targeting score (TTS) system (Guettler, S. et al. Structural basis and sequence rules for substrate recognition by Tankyrase explain the basis for cherubism disease. Cell 147, 1340-1354, doi:10.1016/j.cell.2011.10.046 (2011)). Ingenuity pathway analysis (IPA) revealed four candidate proteins above the TTS cutoff 0.385) that fell into the chondrogenesis category (FIG. 3c). Then IUPred disorder score (Dosztanyi, Z., Csizmok, V., Tompa, P. & Simon, I. IUPred: web server for the prediction of intrinsically unstructured regions of proteins based on estimated energy content. Bioinformatics 21, 3433-3434, doi:10.1093/bioinformatics/bti541 (2005)) was used to filter unlikely targets, wherein tankyrase-binding motifs are positioned in a highly structured region (FIG. 3d). SOX9 exhibited both high TTS and disorder scores, and selected as a candidate. Endogenous interactions between tankyrase and SOX9 in chondrocytes were confirmed by co-immunoprecipitation assay and in situ proximity ligation assay (PLA) (FIG. 3e, f).

Moreover, our cell-based assay indicated that SOX9 binds to both tankyrase-1 and tankyrase-2 (FIG. 3g). The two tankyrase-binding domains (TBDs) of SOX9, designated as TBD1 and TBD2, are highly conserved among vertebrates (FIG. 3h). Based on structural simulations, TBD1 and TBD2 peptides fit into the binding pocket located central to the ankyrin repeat cluster (ARC) IV domain of tankyrase where known substrates, SH3 domain-binding protein (3BP2) and myeloid cell leukemia sequence 1 protein (MCL1), are aligned (FIG. 3i). The deletion of either TBD1 or TBD2 resulted in the reduction in the binding affinity of SOX9 for tankyrase (FIG. 3j), while simultaneous deletion of both TBDs nearly abolished this association (FIG. 3k).

### Example 3. Tankyrase Inhibition Enhances SOX9 Stability and Activity by Uncoupling SOX9 from PARylation-Dependent Degradation

Here, we investigated whether tankyrase binding to SOX9 is coupled to PARylation of SOX9. Wild-type SOX9 underwent extensive PARylation, whereas SOX9 mutant missing both TBDs exhibited a markedly reduced PARylation level (FIG. 3l). Tankyrase-dependent PARylation is generally linked to the degradation of substrate proteins (Riffell, J. L., Lord, C. J. & Ashworth, A. Tankyrase-targeted therapeutics: expanding opportunities in the PARP family. Nat Rev Drug Discov 11, 923-936, doi:10.1038/nrd3868 (2012)). In fact, tankyrase inhibition promoted SOX9 protein expression in chondrocytes (FIG. 3m, n). and SOX9 TBD mutant showed augmented stability compared with wild-type SOX9 (FIG. 3o). Taken together, the disruption of the physical interactions between tankyrase and SOX9 and the consequent abolishment of SOX9 PARylation results in the stabilization of SOX9.

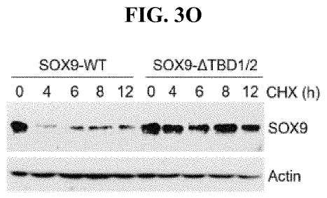

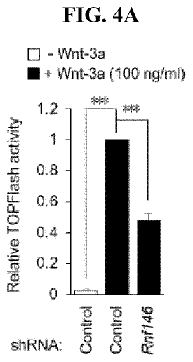

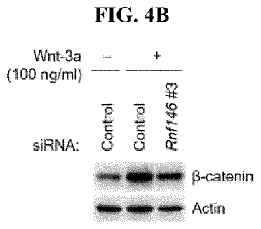

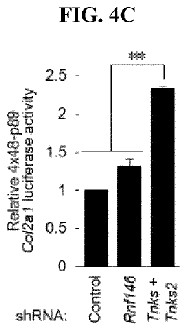

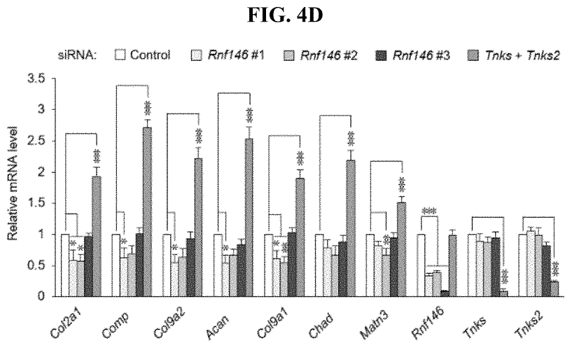

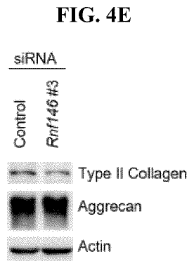

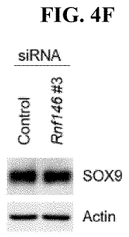

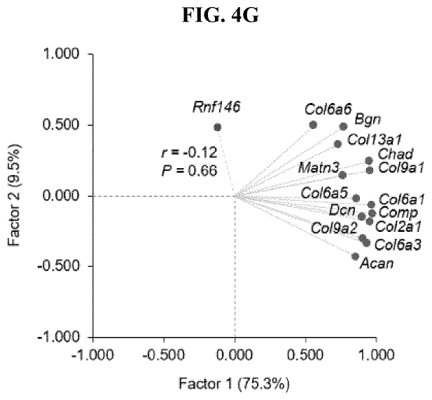

To date, RNF146 is the only known E3 ubiquitin ligase that mediates PARylation-dependent ubiquitination and degradation of substrates (Zhang, Y. et al. RNF146 is a poly(ADP-ribose)-directed E3 ligase that regulates axin degradation and Wnt signalling. Nat Cell Biol 13, 623-629, doi:10.1038/ncb2222 (2011), DaRosa, P. A. et al. Allosteric activation of the RNF146 ubiquitin ligase by a poly(ADP-ribosyl)ation signal. Nature 517, 223-226, doi:10.1038/nature13826 (2015), Andrabi, S. A. et al. Iduna protects the brain from glutamate excitotoxicity and stroke by interfering with poly(ADP-ribose) polymer-induced cell death. Nat Med 17, 692-699, doi:10.1038/nm.2387 (2011)). In particular, RNF146 is best known to regulate tankyrase-dependent Axin degradation and hence, β-catenin stabilization³³. Consistent with this notion, shRNA or siRNA-mediated knockdown of Rnf146 effectively reduced TOPFlash activity and β-catenin level (FIG. 4a, b). However, unlike Tnks and Tnks2 double knockdown, Rnf146 knockdown in chondrocytes failed to increase SOX9 transcriptional activity, the expression of cartilage matrix genes, or SOX9 protein level (FIG. 4c-f). Our experimental findings were further supported by factor analysis results based on mouse reference populations. A total of 14 inter-correlated cartilage matrix genes exhibited insignificant correlation with Rnf146 (r=−0.12; P=0.66; FIG. 4g). Our data suggest an intriguing possibility that PAR-dependent E3 ligases other than RNF146 may exist and regulate PARylation-dependent SOX9 regulation.

### Example 4. Identification that SOX9 is Necessary for Tankyrase Inhibition-Induced Cartilage Matrix Gene Expression

Here, we used a 4×48-p89 SOX9-dependent Col2a1 enhancer reporter (Murakami, S., Lefebvre, V. & de Crombrugghe, B. Potent inhibition of the master chondrogenic factor Sox9 gene by interleukin-1 and tumor necrosis factor-alpha. J Biol Chem 275, 3687-3692 (2000)) to investigate whether the increase in SOX9 levels with tankyrase inhibition enhances the overall transcriptional activity of SOX9. Double knockdown of Tnks and Tnks2 and nine different tankyrase-specific inhibitors specifically increased the transcriptional activity of SOX9 in chondrocytes (FIG. 5a-c). Moreover, the overexpression of wild-type TNKS2 resulted in a marked reduction in the transcriptional activity of SOX9, while the catalytically inactive form of TNKS2 (TNKS2 M1054V) suppressed SOX9 activity to a moderate extent (FIG. 5d).

SOX9 target genes (Oh, C. D. et al. SOX9 regulates multiple genes in chondrocytes, including genes encoding ECM proteins, ECM modification enzymes, receptors, and transporters. PLoS One 9, e107577, doi:10.1371/journal.pone.0107577 (2014)) were overall upregulated upon tankyrase knockdown or inhibition at the whole transcriptome level (FIG. 5e).

Meanwhile, SOX9 is known to bind to its own enhancer and auto-regulate its expression (Mead, T. J. et al. A far-upstream (−70 kb) enhancer mediates Sox9 auto-regulation in somatic tissues during development and adult regeneration. Nucleic Acids Res 41, 4459-4469, doi:10.1093/nar/gkt140 (2013)). As disclosed hereinbefore, we thought that Tankyrase are involved in the degradation of SOX0, we further investigated whether tankyrase regulates SOX9 activity post-transcriptionally at the protein level. For this, the effect of tankyrase inhibition with abundant amount of SOX9 protein expressed as FIGS. 5f and 5g was analyzed and the luciferase reporter assays using the SOX9-dependent Col2a1 enhancer construct in cells constitutively expressing SOX9 mRNA under the control of a cytomegalovirus (CMV) promoter were performed. Tankyrase inhibition using siRNAs or drugs increased the transcriptional activity of exogenously expressed SOX9 in HEK293T cells (FIG. 5f, g). Furthermore, point mutations of Arg in the first position to Ala in both TBD1 and TBD2 of SOX9 synergistically enhanced the transcriptional activity of SOX9 (FIG. 5h), suggesting that disruption of the interaction between tankyrase and SOX9 is sufficient to enhance the transcriptional activity of SOX9. Cartilage matrix gene expression induced by tankyrase inhibition was completely abolished by SOX9 knockdown (FIG. 5i, j). Taken together, SOX9 serves as an essential target of tankyrase for the role of tankyrase as an anabolic regulator in chondrocytes.

### Example 5. Tankyrase Inhibition Protects Against Osteoarthritic Cartilage Destruction in Mice

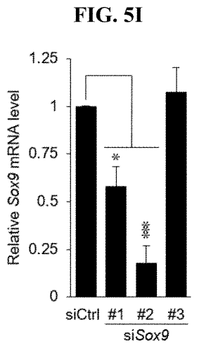

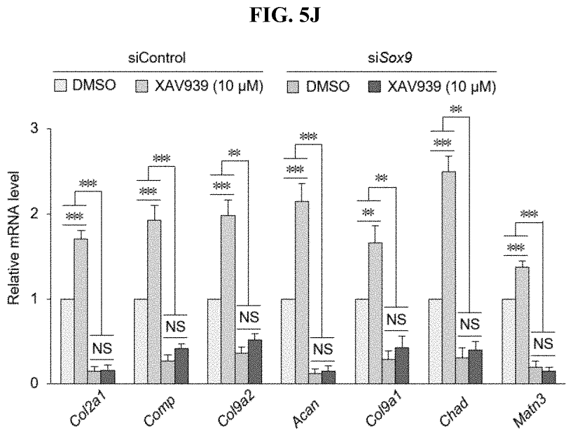

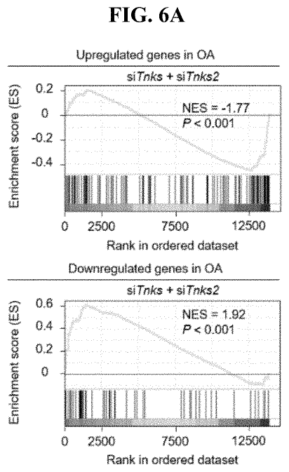

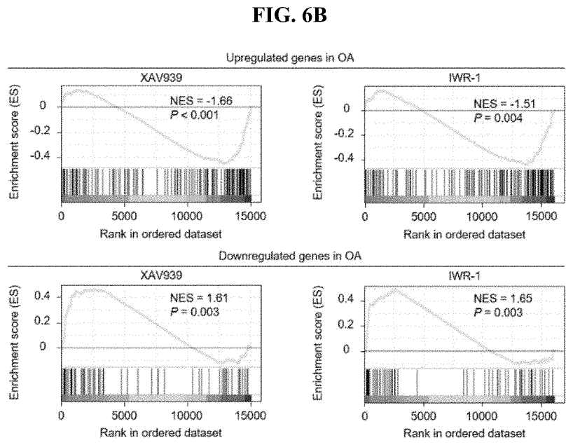

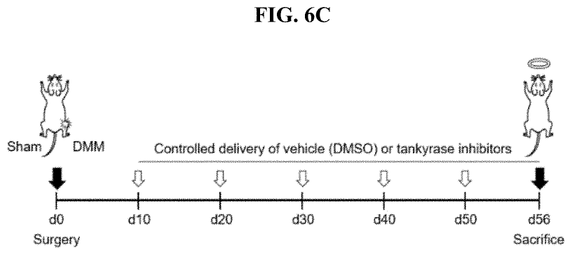

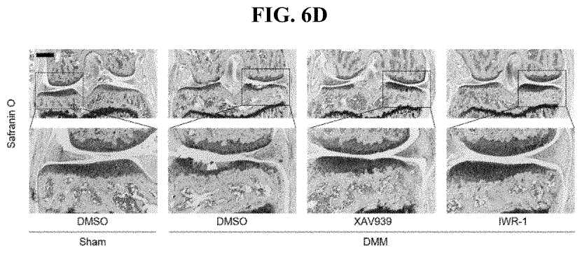

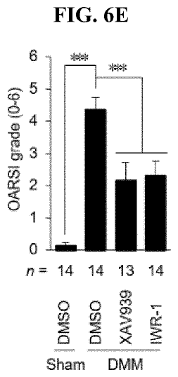

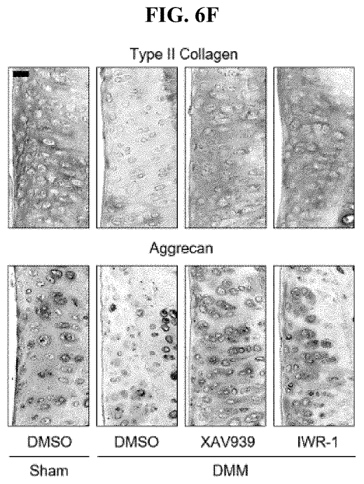

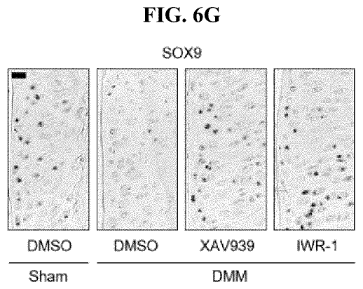

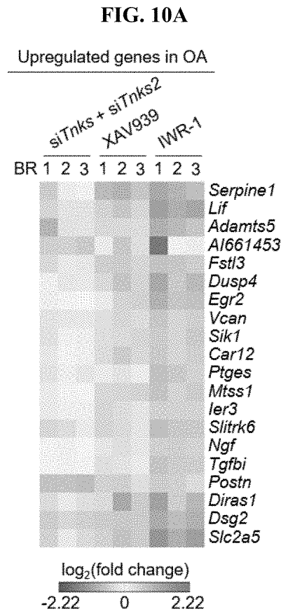

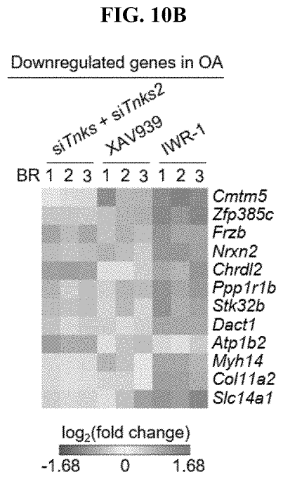

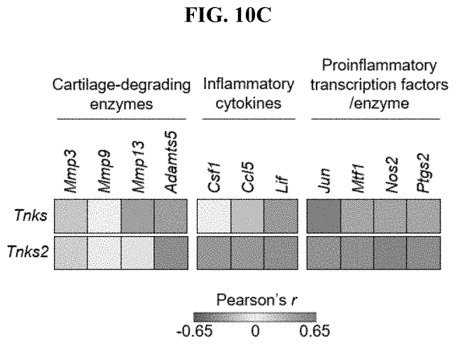

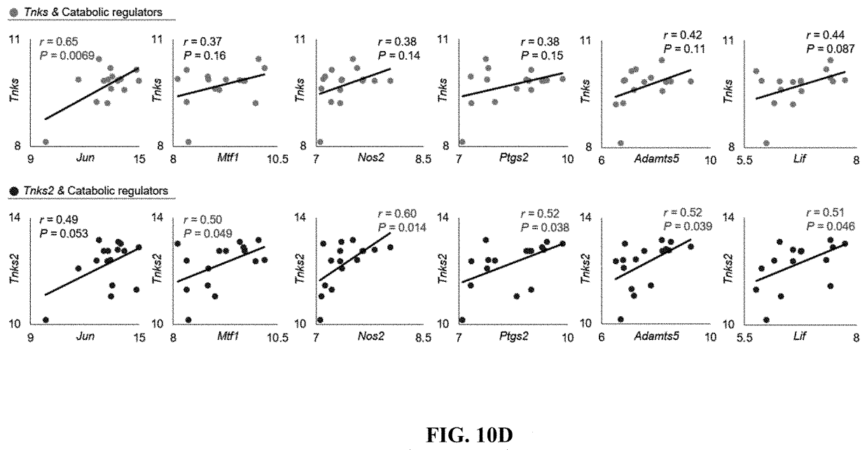

Our results disclosed herein suggest that tankyrase may perform a physiological role in the regulation of cartilage matrix homeostasis. As cartilage homeostasis is disrupted during OA development. Thus, we investigated how tankyrase inhibition affects the expression of OA-associated genes when cartilage matrix homeostasis is destructed during OA development. By utilizing public transcriptome datasets, we generated a comprehensive list of OA-associated genes that are upregulated and downregulated in OA patients. Notably, OA-associated genes upregulated in patients were overall repressed in chondrocytes upon tankyrase inhibition (FIG. 10a). In contrast, OA-associated genes suppressed in patients were strongly transactivated by tankyrase inhibition (FIG. 10b). This inverted pattern of gene expression profiling was evident even at the whole transcriptome level (FIG. 6a, b).

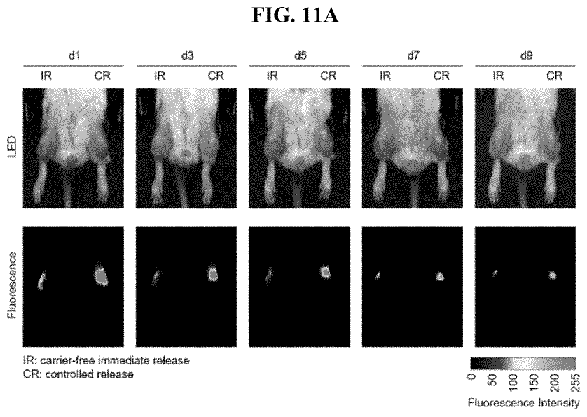

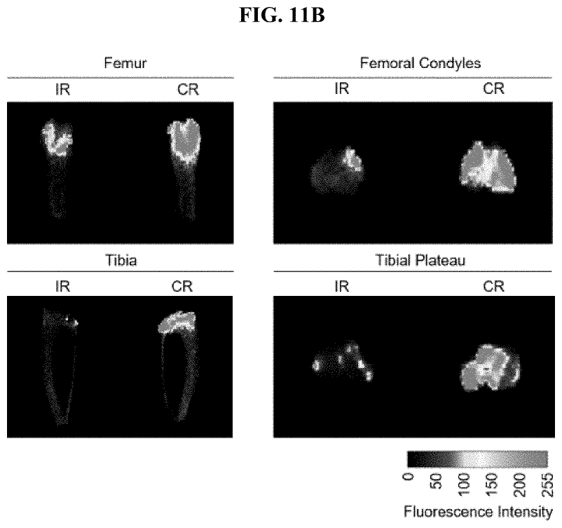

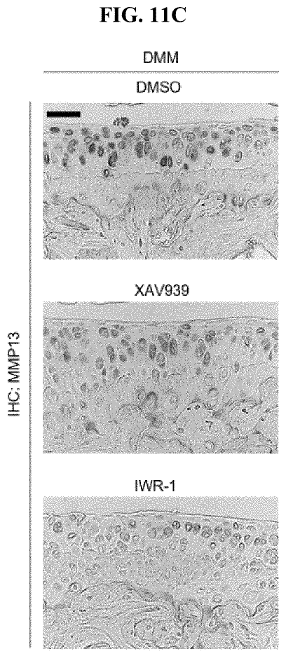

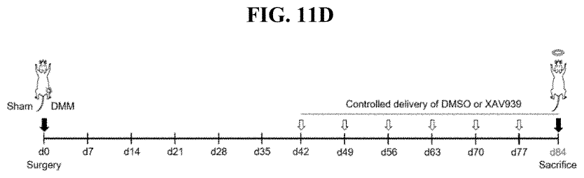

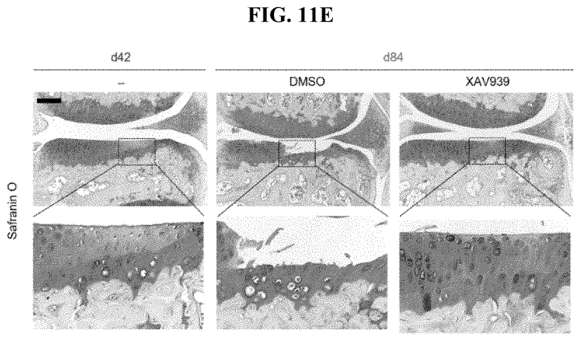

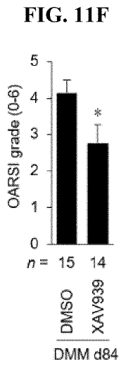

Next, we assessed the in vivo effects of tankyrase inhibition on cartilage matrix homeostasis in surgically induced OA mouse model. For the stable and prolonged delivery of tankyrase inhibitors to mouse knee joints, we used injectable hydrogels made of ascorbyl palmitate. Intra-articular (IA) injection of this hydrogel-based drug delivery system allowed controlled local release of the loaded small molecule to articular cartilage over 9 days (FIG. 11a, b). IA administration of hydrogel-mediated XAV939 or IWR-1, the two representative tankyrase inhibitors with different modes of actions, resulted in a significant reduction in the degeneration of cartilage matrix caused by the destabilization of the medial meniscus (DMM) (FIG. 6c, d, e). A concomitant increase in type II collagen and aggrecan was observed (FIG. 60 and the expression of SOX9 was retained in the cartilage treated with tankyrase inhibitors (Fig. g). In addition, we observed that IA delivery of tankyrase inhibitors effectively reduced the production of matrix metalloproteinase 13 (MMP13)(Billinghurst, R. C. et al. Enhanced cleavage of type II collagen by collagenases in osteoarthritic articular cartilage. J Clin Invest 99, 1534-1545, doi:10.1172/JCI119316 (1997) that is a key enzyme involved in the catabolism of Type II collagen. These experimental results are in line with the correlation analysis based on mouse reference populations, indicating that tankyrase exhibits a negative and positive correlation with cartilage matrix genes (FIG. 1b, c) and catabolic regulators (FIG. 10d, e), respectively.

Based on the pro-anabolic effect of tankyrase inhibitors, we tested the potential of XAV939 to treat late-stage OA cartilage. In the mouse DMM model (Kim, J. H. et al. Matrix cross-linking-mediated mechanotransduction promotes posttraumatic osteoarthritis. Proc Natl Acad Sci USA 112, 9424-9429, doi:10.1073/pnas.1505700112 (2015), early osteoarthritic lesions were observed 2 weeks after surgery, while 70% of mice had reached late-stage OA after 6 weeks from DMM surgery. XAV939 administration for additional 6 weeks resulted in the reduction in the cartilage destruction as compared with the vehicle-treated mice, which experienced further OA progression (FIG. 11c, d, e). Taken together, our results indicate that tankyrase inhibitors effectively ameliorate cartilage destruction in mice through the attenuation of the imbalance between matrix anabolism and catabolism.

### Example 6. Tankyrase Inhibition Stimulates Chondrogenic Differentiation of MSCs and Produce Therapeutic Effects

As mesenchymal progenitor cells are responsible for the regenerative capacity of damaged cartilage (Johnson, K. et al. A stem cell-based approach to cartilage repair. Science 336, 717-721, doi:10.1126/science.1215157 (2012), Jiang, Y. & Tuan, R. S. Origin and function of cartilage stem/progenitor cells in osteoarthritis. Nat Rev Rheumatol 11, 206-212, doi:10.1038/nrrheum.2014.200 (2015)), we investigated the role of tankyrase in the chondrogenic differentiation of MSCs. The tankyrase inhibitors, XAV939 and IWR-1, effectively induced chondrogenic nodule formation in micromass cultures of mouse limb-bud mesenchymal cells (FIG. 7a), and both pharmacological inhibition and double knockdown of TNKS and TNKS2 effectively enhanced the chondrogenic differentiation of hMSCs (FIG. 7b, c, d).

We next evaluated the effect of tankyrase inhibition on stem cell-based restoration of hyaline cartilage. A full-thickness osteochondral lesion was filled with a fibrin gel containing hMSCs transduced with control or TNKS and TNKS2 shRNAs. After 8 weeks, Defects transplanted with hMSCs-control shRNA failed to fully recover the organization of hyaline cartilage and exhibited features of fibrocartilage (FIG. 7e, f and FIG. 12). However, lesions implanted with hMSCs-shTNKS/2 showed regenerated hyaline cartilage, similar to the articular cartilage with robust expression of SOX9 and cartilage-specific matrix proteins (FIG. 7g, h).

Innate MSCs are present in cartilage tissues and there are many MSCs in the bone marrow and synovial fluid around the cartilage, which may be involved in cartilage regeneration. Here it was shown that the inhibition of Tankyrase can lead to the differentiation of MSCs into chondrocytes in cell and mouse cartilage regeneration model. This indicates that the promotion of differentiation of MSC into chondrocytes by inhibition of Tankyrase can be advantageously used for cartilage regeneration in degenerative arthritis.

Unless defined otherwise, all technical and scientific terms and any acronyms used herein have the same meanings as commonly understood by one of ordinary skill in the art in the field of the invention. Although any methods and materials similar or equivalent to those described herein can be used in the practice of the present invention, the preferred methods, devices, and materials are described herein.

1. A method of treating arthritis in a subject in need thereof comprising administering to the subject an effective amount of an inhibitor of Tankyrase; or an modified adult stem cell in which the expression of Tankyrase is suppressed or Tankyrase gene is knocked out,
wherein the inhibitor of Tankyrase or the modified adult stem cell stabilizes the Sox9 protein or increases the concentration of the Sox9 protein by inhibiting the Tankyrase activity promoting the degradation of Sox9 protein.

2. The method of claim 1, wherein the inhibitor of Tankyrase leads to a chondrogenic differentiation of an adult stem cells leading to chondrogenic regeneration.

3. The method of claim 1, wherein the inhibitor of Tankyrase is an agent that binds to a nicotinamide sub-domain of ARTD domain which is a catalytic domain of a Tankyrase protein, an agent that binds to an adenosine sub-domain of a Tankyrase protein or an agent that binds to an unidentified domain of a Tankyrase protein.

4. The method of claim 3,
wherein the agent that binds to a nicotinamide sub-domain of ARTD domain is XAV939 {3,5,7,8-tetrahydro-2-[4-(trifluoromethyl)phenyl]-4H-thiopyrano[4,3-d]pyrimidine-4-one} or MN-64 {2-[4-(1-methylethyl)phenyl]-4H-1-benzopyran-4-one};
the agent that binds to an adenosine sub-domain of a Tankyrase protein is IWR-1 [4-(1,3,3a,4,7,7a-hexahydro-1,3-dioxo-4,7-methano-2H-isoindole-2-yl)-N-8-quinolynyl-benzam ide], JW55 {N-[4-[[[[tetrahydro-4-(4-methoxyphenyl)-2H-pyran-4-yl]methyl]amino]carbonyl]phenyl]-2-purancarboxamide}, WIKI4 2-[3-[[4-(4-methoxyphenyl)-5-(4-pyridynyl)-4H-1,2,4-triazol-3-yl]thio]propyl]-1Hbenz[de]isoquinoline-1,3(2H)-dion, TC-E5001 {3-(4-methoxyphenyl)-5-[[[4-(4-methoxyphenyl)-5-methyl-4H-1,2,4-triazol-3-yl]thio]methyl]-1,2,4-oxadiazol or G007-LK {(E)-4-(5-(2-(4-(2-chlorophenyl)-5-(5-(methylsulfonyl)pyridine-2-yl)-4H-1,2,4-triazol-3-yl)vinyl)-1,3,4-oxadiazole-2-yl)benzonitrile}; and
the agent that binds to an unidentified domain of a Tankyrase protein is G244-LM {3,5,7,8-tetrahydro-2-[4-[2-(methylsulfonyl) phenyl]-1-piperazynyl]-4H-thiopyrano[4,3-d]pyrimidine-4-one}, or AZ6102 {rel-2-[4-[6-[(3R,5S)-3,5-dimethyl-1-piperazynyl]-4-methyl-3-pyridynyl]phenyl]-3,7-dihydro-7-methyl-4H-pyrrolo[2,3-d]pyrimidine-4-one}, or isomers or derivative thereof.

5. The method of claim 1,
wherein the inhibitor is a siRNA that suppresses the expression of Tankyrase gene into a Tankyrase protein.

6. The method of claim 5,
wherein the siRNA is a dsRNA consisting of RNAs of SEQ ID NO: X1 and SEQ ID NO:X2; or a dsRNA consisting of RNAs of SEQ ID NO: X3 and SEQ ID NO:X4.

7. The method of claim 1,
wherein the adult stem cell is autologous or allogenic.

8. The method of claim 1,
wherein the adult stem cell is a mesenchymal stem cell.

9. A method of promoting the differentiation of an adult stem cell into a cartilage cell by treating the stem cell with an inhibitor of Tankyrase.

10. The method of claim 9,
wherein the adult stem cell is a mesenchymal stem cell.

11. The method of claim 2, wherein the inhibitor of Tankyrase is an agent that binds to a nicotinamide sub-domain of ARTD domain which is a catalytic domain of a Tankyrase protein, an agent that binds to an adenosine sub-domain of a Tankyrase protein or an agent that binds to an unidentified domain of a Tankyrase protein.

12. The method of claim 11,
wherein the agent that binds to a nicotinamide sub-domain of ARTD domain is XAV939 {3,5,7,8-tetrahydro-2-[4-(trifluoromethyl)phenyl]-4H-thiopyrano[4,3-d]pyrimidine-4-one} or MN-64 {2-[4-(1-methylethyl)phenyl]-4H-1-benzopyran-4-one};
the agent that binds to an adenosine sub-domain of a Tankyrase protein is IWR-1 [4-(1,3,3a,4,7,7a-hexahydro-1,3-dioxo-4,7-methano-2H-isoindole-2-yl)-N-8-quinolynyl-benzam ide], JW55 {N-[4-[[[[tetrahydro-4-(4-methoxyphenyl)-2H-pyran-4-yl]methyl]amino]carbonyl]phenyl]-2-purancarboxamide}, WIKI4 2-[3-[[4-(4-methoxyphenyl)-5-(4-pyridynyl)-4H-1,2,4-triazol-3-yl]thio]propyl]-1Hbenz[de]isoquinoline-1,3(2H)-dion, TC-E5001 {3-(4-methoxyphenyl)-5-[[[4-(4-methoxyphenyl)-5-methyl-4H-1,2,4-triazol-3-yl]thio]methyl]-1,2,4-oxadiazol or G007-LK {(E)-4-(5-(2-(4-(2-chlorophenyl)-5-(5-(methylsulfonyl)pyridine-2-yl)-4H-1,2,4-triazol-3-yl)vinyl)-1,3,4-oxadiazole-2-yl)benzonitrile}; and
the agent that binds to an unidentified domain of a Tankyrase protein is G244-LM {3,5,7,8-tetrahydro-2-[4-[2-(methylsulfonyl) phenyl]-1-piperazynyl]-4H-thiopyrano[4,3-d]pyrimidine-4-one}, or AZ6102 {rel-2-[4-[6-[(3R,5S)-3,5-dimethyl-1-piperazynyl]-4-methyl-3-pyridynyl]phenyl]-3,7-dihydro-7-methyl-4H-pyrrolo[2,3-d]pyrimidine-4-one}, or isomers or derivative thereof.

13. The method of claim 2,
wherein the inhibitor is a siRNA that suppresses the expression of Tankyrase gene into a Tankyrase protein.

14. The method of claim 13,
wherein the siRNA is a dsRNA consisting of RNAs of SEQ ID NO: X1 and SEQ ID NO:X2; or a dsRNA consisting of RNAs of SEQ ID NO: X3 and SEQ ID NO:X4.

15. The method of claim 2.
wherein the adult stem cell is autologous or allogenic.

16. The method of claim 2.
wherein the adult stem cell is a mesenchymal stem cell.
